(19) United States
(12) Patent Application Publication
(10) Pub. No.: US 2026/0357328 A1
(43) Pub. Date: Nov. 12, 2026

(54) OLIGONUCLEOTIDE PROBES FOR DETECTING TARGET ALLELES

(71) Applicant: Integrated DNA Technologies, Inc., Coralville, IA (US)

(72) Inventor: Richard Owczarzy, Coralville, IA (US)

(21) Appl. No.: 19/313,327

(22) Filed: Aug. 28, 2025

Related U.S. Application Data
(60) Provisional application No. 63/689,241, filed on Aug. 30, 2024.
(60) Provisional application No. 63/735,135, filed on Dec. 17, 2024.

(51) Int. Cl. C12Q 1/6827; C12Q 1/6806; C12Q 1/6823; C12Q 1/6858

(52) U.S. Cl. CPC C12Q 1/6827; C12Q 1/6806; C12Q 1/6823; C12Q 1/6858

(57) ABSTRACT

An oligonucleotide probe for detecting a genomic alteration includes a first complementary region, a second complementary region, and a semi-complementary region. The first complementary region includes at least three nucleotides that are completely complementary to a mutant allele of a portion of a genomic sequence, the includes at least three nucleotides that are completely complementary to the mutant allele and to the wild type allele, and the semi-complementary region is adjacently flanked by the first complementary region and the second complementary region. The nucleotides of the semi-complementary region are completely complementary to the mutant allele and are not completely complementary to the wild type allele, the first complementary region comprises a first locked nucleic acid adjacent to the semi-complementary region, the second complementary region comprises a second locked nucleic acid adjacent to the semi-complementary region, and the semi-complementary region comprises a third locked nucleic acid.

### CROSS-REFERENCE TO RELATED APPLICATION(S)

This application claims the benefit of U.S. Provisional Application No. 63/689,241, filed Aug. 30, 2024, and entitled “DESIGN RULES FOR OLIGONUCLEOTIDE PROBES WITH IMPROVED MELTING TEMPERATURE DISCRIMINATION,” and claims the benefit of U.S. Provisional Application No. 63/735,135, filed Dec. 17, 2024, and entitled “OLIGONUCLEOTIDE PROBES FOR DETECTING TARGET ALLELES,” the disclosures of which are hereby incorporated by reference in their entirety.

### FIELD OF THE INVENTION

The present disclosure relates to oligonucleotide probes and, more particularly, oligonucleotide probes for detecting specific alleles and methods of using oligonucleotide probes to detect specific alleles.

### REFERENCE TO AN ELECTRONIC SEQUENCE LISTING

The present application includes a Sequence Listing which has been submitted electronically as an XML document in the ST.26 format via Patent Center and concurrent with the filing of the application, and which is hereby incorporated by reference in its entirety. The XML document is named “ID2024-D0487-P15836US04.xml,” was created on Aug. 22, 2025, and is 238,743 bytes in size.

### BACKGROUND

Oligonucleotides are short, synthetically-generated oligomers of deoxyribonucleic acid (DNA) moieties, ribonucleic acid moieties (RNA), or a mixture thereof. Oligonucleotides can be manufactured as single-stranded molecules with a custom, user-specified sequence. Oligonucleotide probes can be constructed for detecting genomic variants and/or alterations to the genome, such as mutations.

### SUMMARY

An example of an oligonucleotide probe for detecting a genomic alteration includes a first complementary region, a second complementary region, and a semi-complementary region. The first complementary region includes at least three nucleotides that are completely complementary to a mutant allele of a portion of a genomic sequence, the includes at least three nucleotides that are completely complementary to the mutant allele and to the wild type allele, and the semi-complementary region is adjacently flanked by the first complementary region and the second complementary region. The nucleotides of the semi-complementary region are completely complementary to the mutant allele and are not completely complementary to the wild type allele, the first complementary region comprises a first locked nucleic acid adjacent to the semi-complementary region, the second complementary region comprises a second locked nucleic acid adjacent to the semi-complementary region, and the semi-complementary region comprises a third locked nucleic acid.

A further example of an oligonucleotide probe for detecting a genomic alteration includes a first complementary region, a second complementary region, and a semi-complementary region. The first complementary region includes at least three nucleotides that are completely complementary to a mutant allele of a portion of a genomic sequence and to a wild type allele of the portion of the genomic sequence, the second complementary region includes at least three nucleotides that are completely complementary to the mutant allele and to the wild type allele, and the semi-complementary region includes three locked nucleic acid residues that are completely complementary to the mutant allele and that are not completely complementary to the wild type allele.

An example of a method of detecting a mutant allele of a gene includes providing a sample containing the mutant allele and a wildtype allele of the gene, providing the sample with an oligonucleotide probe, heating the sample to denature the mutant allele and the wildtype allele, lowering the temperature of the sample, after heating the sample, to an annealing temperature below a first melting temperature between the oligonucleotide probe and the mutant allele and above a second melting temperature between the oligonucleotide probe and the wild type allele, hybridizing the probe to the mutant allele at the annealing temperature to form a mutant allele and oligonucleotide probe hybrid, and detecting the mutant allele by detecting the mutant allele and oligonucleotide probe hybrid. The oligonucleotide probe includes a first complementary region, a second complementary region, and a semi-complementary region. The first complementary region includes at least three nucleotides that are completely complementary to a mutant allele of a portion of a genomic sequence, the includes at least three nucleotides that are completely complementary to the mutant allele and to the wild type allele, and the semi-complementary region is adjacently flanked by the first complementary region and the second complementary region. The nucleotides of the semi-complementary region are completely complementary to the mutant allele and are not completely complementary to the wild type allele, the first complementary region comprises a first locked nucleic acid adjacent to the semi-complementary region, the second complementary region comprises a second locked nucleic acid adjacent to the semi-complementary region, and the semi-complementary region comprises a third locked nucleic acid.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1A is a schematic diagram of an example of an oligonucleotide probe duplex that includes a bulged region.

FIG. 1B is a schematic diagram of an example of a duplex including the oligonucleotide probe of FIG. 1A and not including a bulged region.

FIG. 2A is a schematic diagram of an example of an oligonucleotide probe duplex that includes a bulged region.

FIG. 2B is a schematic diagram of an example of a duplex including the oligonucleotide probe of FIG. 2A and not including a bulged region.

FIG. 3A is a schematic diagram of an example of an oligonucleotide probe duplex that includes a mismatched region.

FIG. 3B is a schematic diagram of an example of a duplex including the oligonucleotide probe of FIG. 3A and not including a mismatched region.

FIG. 4 is a graph of discrimination as a function of location of a bulge or mismatch.

FIG. 5 is a schematic diagram of an example of a design for a locked nucleic acid-containing oligonucleotide probe according to the present disclosure.

FIG. 6 is a schematic diagram of another example of a design for a locked nucleic acid-containing oligonucleotide probe according to the present disclosure.

FIG. 7 is a chemical diagram of an example of a minor groove binder moiety.

FIG. 8 is a schematic diagram of an example of a design for a minor groove binder-containing oligonucleotide probe according to the present disclosure.

FIG. 9 is a flow diagram of an example of a method of detecting a genomic alteration or variation.

FIG. 10 is a flow diagram of another example of a method of detecting a genomic alteration or variation.

While the above-identified figures set forth one or more examples of the present disclosure, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features and components not specifically shown in the drawings.

### DETAILED DESCRIPTION

The present disclosure relates to oligonucleotides probes for detecting specific alleles of biallelic or multiallelic genomic regions and methods for detecting specific alleles of alleles of biallelic or multiallelic genomic regions using the oligonucleotide probes disclosed herein. In particular, the present disclosure provides specific design principles (and data supporting those design principles) that can be used to create oligonucleotide probes containing locked nucleic acid (LNA) moieties or minor groove binder (MGB) moieties. LNA-containing and MGB-containing oligonucleotide probes designed according to the present disclosure have improved discrimination as compared to unmodified oligonucleotide probes and, accordingly, have improved specificity when used in methods of specific allele detection, such as 5′ nuclease assays.

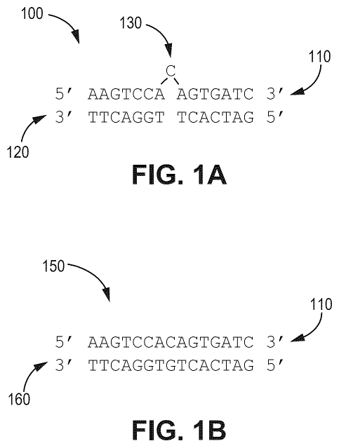

FIGS. 1A-3B are examples of oligonucleotide probe duplexes and highlight different structures that form between oligonucleotide probes and target alleles.

FIG. 1A is a schematic depiction of oligonucleotide probe duplex 100 and FIG. 1B is a schematic depiction of oligonucleotide probe duplex 150. FIGS. 1A and 1B are discussed together herein. Oligonucleotide probe duplexes 100 and 150 are examples of oligonucleotide probe duplexes according to the present disclosure. Oligonucleotide probe duplex 100 includes oligonucleotide probe 110 and genomic DNA segment 120. Probe 110 is hybridized to genomic DNA segment 120 in oligonucleotide probe duplex 100 but is only partially complementary to genomic DNA segment 120, causing the formation of bulged region 130. Oligonucleotide probe duplex 150 also includes oligonucleotide probe 110, but in oligonucleotide probe duplex 150, oligonucleotide probe 110 is hybridized to genomic DNA segment 160. Oligonucleotide probe 110 is completely complementary to genomic DNA segment 160 and, accordingly, no bulges or mismatches are formed in oligonucleotide probe duplex 150. Oligonucleotide probe 110 preferentially binds to genomic DNA segment 160 over genomic DNA segment 120 and, accordingly, the melting temperature of oligonucleotide probe duplex 150 is higher than the melting temperature of oligonucleotide probe duplex 100. The difference in melting temperature between two related oligonucleotide probe duplexes, such as oligonucleotide probe duplex 100 and oligonucleotide probe duplex 150, is generally referred to herein as “discrimination.”

Notably, genomic DNA segment 160 and oligonucleotide probe 110 include one nucleotide more than genomic DNA segment 120. As such, oligonucleotide probe 110 forms bulge region 130 when duplexed with genomic DNA segment 120. Genomic DNA segment 160 can belong to, for example, a mutant allele containing a single-nucleotide insertion and genomic DNA segment 120 can belong to a corresponding wildtype allele. Additionally and/or alternatively, genomic DNA segment 160 can belong to a wildtype allele and genomic DNA segment 120 can belong to a corresponding mutant allele including a single-nucleotide deletion. Bulge region 130 can be of any size but is depicted as a single nucleotide for explanatory convenience.

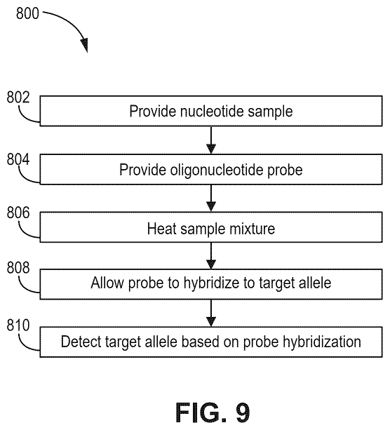

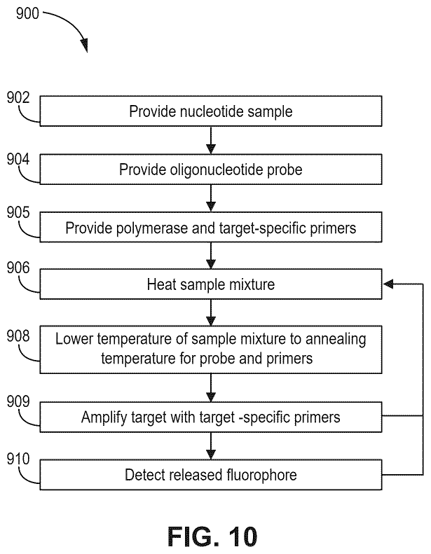

The difference in melting temperature between oligonucleotide probe duplex 150 and oligonucleotide probe duplex 100 enables oligonucleotide probe 110 to bind to genomic DNA segment 160 at a higher temperature than the melting temperature of oligonucleotide probe duplex 100, thereby enabling oligonucleotide probe 110 to be used to selectively detect of genomic DNA segment 160 using, for example, method 800 and/or method 900 (described in more detail subsequently with respect to FIG. 9 and FIG. 10, respectively).

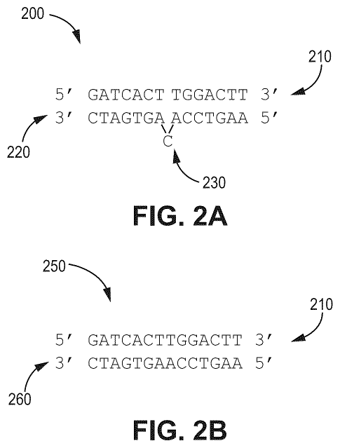

FIG. 2A is a schematic depiction of oligonucleotide probe duplex 200 and FIG. 2B is a schematic depiction of oligonucleotide probe duplex 250. FIGS. 2A and 2B are discussed together herein. Oligonucleotide probe duplexes 200 and 250 are further examples of oligonucleotide probe duplexes according to the present disclosure. Oligonucleotide probe duplex 200 includes oligonucleotide probe 210 and genomic DNA segment 220. Probe 210 is hybridized to genomic DNA segment 220 in oligonucleotide probe duplex 200 but is only partially complementary to genomic DNA segment 220. The non-complementary portion of genomic DNA segment 220 forms bulged region 230. Like oligonucleotide probe duplex 200, oligonucleotide probe duplex 250 also includes oligonucleotide probe 210. However, in oligonucleotide probe duplex 250, oligonucleotide probe 210 is hybridized to genomic DNA segment 260 rather than genomic DNA segment 220. Oligonucleotide probe 210 is completely complementary to genomic DNA segment 260 and no bulges or mismatches are formed in oligonucleotide probe duplex 250. Because oligonucleotide probe 210 is completely complementary to genomic DNA segment 260 and only partially complementary to genomic DNA segment 220, oligonucleotide probe 210 preferentially binds to genomic DNA segment 260 over genomic DNA segment 220 and the melting temperature of oligonucleotide probe duplex 250 is higher than the melting temperature of oligonucleotide probe duplex 200.

Genomic DNA segment 260 and oligonucleotide probe 210 include one nucleotide less than genomic DNA segment 220. As such, genomic DNA segment 220 forms bulge region 230 when duplexed with oligonucleotide probe 210. Genomic DNA segment 260 can belong to, for example, a mutant allele containing a single-nucleotide deletion and genomic DNA segment 220 can belong to a corresponding wildtype allele. Additionally, and/or alternatively, genomic DNA segment 260 can belong to a wildtype allele and genomic DNA segment 220 can belong to a corresponding mutant allele including a single-nucleotide insertion. Bulge region 230 can be of any size but is depicted as a single nucleotide for explanatory convenience.

The difference in melting temperature between oligonucleotide probe duplex 250 and oligonucleotide probe duplex 200 enables oligonucleotide probe 210 to bind to genomic DNA segment 260 at a higher temperature than the melting temperature of oligonucleotide probe duplex 200, thereby enabling oligonucleotide probe 210 to be used to selectively detect genomic DNA segment 260 using, for example, method 800 and/or method 900 (described in more detail subsequently with respect to FIG. 9 and FIG. 10, respectively).

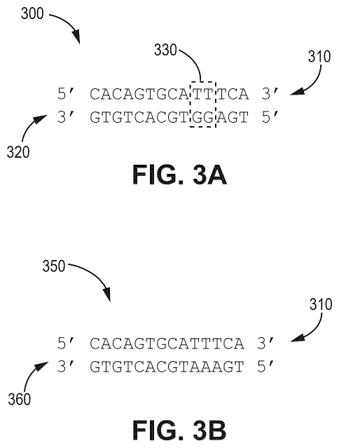

FIG. 3A is a schematic depiction of oligonucleotide probe duplex 300 and FIG. 3B is a schematic depiction of oligonucleotide probe duplex 350. FIGS. 3A and 3B are discussed together herein. Oligonucleotide probe duplexes 300 and 350 are examples of oligonucleotide probe duplexes according to the present disclosure. Oligonucleotide probe duplex 300 includes oligonucleotide probe 310 and genomic DNA segment 320. Probe 310 is hybridized to genomic DNA segment 320 in oligonucleotide probe duplex 300 but is only partially complementary to genomic DNA segment 320, causing the formation of mismatched region 330, which is indicated by a dashed box in FIG. 3A. In some examples, the structure of mismatched region 330 can be referred to as an “internal loop.” Oligonucleotide probe duplex 350 also includes oligonucleotide probe 310, but in oligonucleotide probe duplex 350, oligonucleotide probe 310 is hybridized to genomic DNA segment 360. Oligonucleotide probe 310 is completely complementary to genomic DNA segment 360 and, accordingly, no bulges or internal loops are formed in oligonucleotide probe duplex 350.

Genomic DNA segment 320 and oligonucleotide probe 310 include noncomplementary nucleotides in mismatched region 330. In particular, in mismatched region 330, genomic DNA segment 320 includes two guanosine moieties that are not able to base pair to the corresponding thymidine moieties of oligonucleotide probe 310. Conversely, genomic DNA segment 360 includes a pair of adenosine moieties at the corresponding positions that are able to base pair to the two thymidine moieties of oligonucleotide probe 310. The mismatched residues in oligonucleotide probe duplex 300 causes oligonucleotide probe 310 to preferentially bind to genomic DNA segment 360 and, accordingly, the melting temperature of oligonucleotide probe duplex 350 is higher than the melting temperature of oligonucleotide probe duplex 300.

Genomic DNA segment 360 can belong to, for example, a mutant allele containing a two-nucleotide substitution and genomic DNA segment 320 can belong to a corresponding wildtype allele. Additionally and/or alternatively, genomic DNA segment 360 can belong to a wildtype allele and genomic DNA segment 320 can belong to a corresponding mutant allele including two consecutive substitutions. FIG. 3A depicts a nucleotide duplex having a two-nucleotide mismatch region. However, in other examples, a mismatch region can be of any suitable size, including a single nucleotide or three or more nucleotides.

The difference in melting temperature between oligonucleotide probe duplex 350 and oligonucleotide probe duplex 300 enables oligonucleotide probe 310 to bind to genomic DNA segment 360 at a higher temperature than the melting temperature of oligonucleotide probe duplex 300, thereby enabling oligonucleotide probe 310 to be used to selectively detect genomic DNA segment 360 using, for example, method 800 and/or method 900 (described in more detail subsequently with respect to FIG. 9 and FIG. 10, respectively).

Each of genomic regions 120, 160, 220, 260, 320, 360 can be a coding region of a gene, a noncoding regions of a gene, an extragenic region of a genome, any other suitable region of a genome, and/or any suitable mixture of the foregoing (e.g., a region that overlaps coding and noncoding regions, etc.).

As described above with respect to the discussion of FIGS. 1A-3B, each of oligonucleotide probes 110, 210, and 310 are each able to preferentially bind one genomic DNA segment among related DNA segments that differ by one or more mutations. However, in many examples, the difference in melting temperature between the matched duplex (e.g., one of oligonucleotide probe duplexes 150, 250, 350) and the bulged or mismatched duplex (e.g., one of oligonucleotide probe duplexes 100, 200, 300) can be relatively small (e.g., less than 1 °C).

The present disclosure provides various modified oligonucleotides that have advantageously improved discrimination as compared to equivalent unmodified probes. Example 1, discussed subsequently, provides experimental data relating to advantageous and, in some examples, optimal positions of LNA moieties to increase and/or maximize discrimination. The data of Example 1 is followed by design principles that can be applied to the construction of LNA-containing oligonucleotide probes. Example 2, discussed subsequently, provides experimental data relating to advantageous and, in some examples, optimal positions of mismatch or bulged regions relative to MGB moieties to increase and/or maximize discrimination. The data of Example 2 is followed by design principles that can be applied to the construction of MGB-containing oligonucleotide probes.

### EXAMPLES

The following examples are illustrative and are not intended to limit the scope of the invention.

### Example 1—Design of LNA-Containing Oligonucleotide Probes

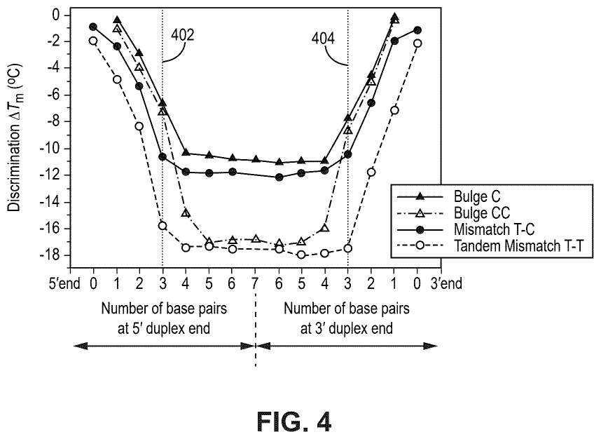

Tables 1-7 and FIG. 4 present data that demonstrates the effect of LNA moieties on oligonucleotide probe discrimination (and, accordingly, the specificity of oligonucleotide probes). As is known, LNAs are modified RNA analogues that include a methyl bridge connecting the 2′ oxygen and the 4′ carbon of the ribose ring.

Tables 1-2 present data that demonstrates the effect of LNA position(s) relative to the position of a bulge within the probe (i.e., in a bulged probe duplex) on discrimination; Tables 3-4 present data that demonstrates the effect of LNA position(s) relative to the position of a bulge in the target sequence (i.e., in a bulged duplex); Tables 5-6 present data that demonstrates the effect of LNA position(s) relative to the position of one or more mismatch nucleotides in a mismatched duplex; Table 7 examines the role of buffer composition on discrimination for LNA-containing oligonucleotides; and FIG. 4 provides data that demonstrates the effect of bulge or mismatch position from the 5′ or 3′ end of a probe on melting temperature.

The data presented in tables 1-7 and FIG. 4 is the basis of the oligonucleotide probe design strategy for LNA-containing oligonucleotides presented subsequently herein. The discrimination data presented in Tables 1-7 is the difference in melting temperature between the reference-matched duplex and one of a bulged duplex and a mismatched duplex, according to the caption of the table and the accompanying description herein. As referred to herein, a “reference-matched” duplex does not contain a bulge, such that the duplex is completely complementary. Conversely, as referred to herein, a “bulged duplex” is only completely complementary at residues other than those indicated as “bulge” nucleotides or “bulged” nucleotides. Moreover, as referred to herein, a “mismatched duplex” is only completely complementary at residues other than those indicated as “mismatched” nucleotides or “mismatch” nucleotides.

The oligonucleotides for which discrimination information is presented in Tables 1-7 include various quantities (i.e., “counts” in the data of Tables 1-6) of LNA residues in various motifs or patterns relative to the bulge position in a bulged duplex (in Tables 1-4) or mismatch position in a mismatched duplex (in Tables 5-6). In particular, the patterns and quantities of LNAs are varied in Tables 1-6 to understand the impact on discrimination of various LNA motifs. In particular, the probes presented in Tables 1-7 generally contain 0, 1, 2, 3, or 4 LNA moieties. A maximum of four LNA moieties are examined in the data of Tables 1-7 due to constraints on the synthesis of LNA-containing oligonucleotides.

The experiments to generate the data provided in Tables 1-7 and FIG. 4 were performed in 1M Na⁺ buffer (1M NaCl, 10 mM sodium phosphate, 1 mM Na₂EDTA, pH 7.0), except for samples indicated in Table 7 as being in Integrated DNA Technologies (IDT) PCR buffer (2.8 mM free Mg²⁺, and 63.3 mM monovalent cations, pH 8.4).

Oligodeoxynucleotides were synthesized, purified by high-performance liquid chromatography (HPLC), and desalted by Integrated DNA Technologies. DNA samples were rehydrated in 10 mM Tris-HCl, 0.1 mM EDTA, pH 7.5 buffer and filtered through a 0.20 μm Milex-LG low binding hydrophilic polytetrafluoroethylene (PTFE) filter. Melting temperature experiments were otherwise performed as specified in Owczarzy R., You Y., Moreira B. G., Manthey J. A., Huang L., Behlke M. A., and Walder J. A., Effects of sodium ions on DNA duplex oligomers: Improved predictions of melting temperatures, Biochemistry 43 (2004) 3537-3554 (hereinafter, “Owczarzy, 2004”). Concentrations of single-strand oligonucleotides were determined from absorbance at 260 nm and predicted extinction coefficients that were calculated using the nearest-neighbor model (Fasman, G. D. (Ed.) (1975) Handbook of Biochemistry and Molecular Biology, Vol. I, p 589, CRC Press, Boca Raton, FL.; Cantor, C. R., Warshaw, M. M., and Shapiro, H. (1970)) Oligonucleotide interactions. III. Circular dichroism studies of the conformation of deoxyoligonucleotides, Biopolymers 9, 1059-1077). Concentrations of oligonucleotides were determined at least twice for each sample using different dilutions. If these concentrations for any sample differed by more than 4%, the results were discarded and measurements were repeated. Complementary oligonucleotides were mixed in 1:1 molar ratios (each strand at 1 μM), heated to 94 °C, and slowly cooled to ambient temperature to form duplexes.

Melting temperature experiments were conducted on a single beam Beckman DU 650 spectrophotometer using 1 centimeter path-length cuvettes. Samples were heated in the range of 10-95 °C with a Micro Tm Analysis accessory and a Beckman High Performance Peltier controller. Absorbance values at 268 nm were recorded every 0.1 °C. Both heating (denaturation) and cooling (renaturation) transition curves were obtained at a controlled rate of temperature change of 24.9 °C/h. Temperatures were measured by an internal probe located inside the Peltier holder and corrected to temperatures of solutions in the cuvettes using calibration function.

Melting profiles of buffers alone were subtracted from the raw absorbance versus temperature profiles of DNA samples. The regions of linear increase of absorbance before and after the melting transition were least-squares fit to lower and upper linear baselines. The fraction of melted base pairs was calculated using the formula, θ=(A−A_{L})/(A_{U}−A_{L}), where A, A_{L}, and A_{U} are sample absorbance, absorbance of the lower baseline, and absorbance of the upper baseline, respectively. Melting temperatures were read at the midpoint of the transitions where θ=0.5 and half of the base pairs are melted. Melting curves collected from 2 different cuvettes were averaged to minimize systematic errors. Average error of melting temperatures was estimated to be 0.4 °C.

Table 1 below includes melting temperature data that demonstrates the effect on oligonucleotide probe discrimination of the relative positioning of LNA residues and bulge nucleotides within the oligonucleotide probes. As used herein, the “Tₘ” symbol refers to duplex melting temperature. Table 1 presents the reference-matched duplex melting temperature as well as bulged duplex melting temperature for various LNA-containing nucleotide probes and various bulge positions. The relative position of the bulge (i.e., in the bulged duplex) and the LNA(s) were varied to understand the impact of LNA quantity, LNA position, and bulge position on oligonucleotide discrimination.

As is described in the caption for Table 1, the location of the bulge nucleotide in the bulged duplex is indicated by an underline. In Table 1, as well as Tables 2-7, LNAs are indicated in the oligonucleotide probe sequences by a “plus” (“+”) symbol prior to the identity of the nucleotide. The oligonucleotide probe sequences presented in Table 1 are grouped according to the identity of the bulge nucleotide, which is indicated by the underlined headings in the “Oligonucleotide Probe Sequence (5′ to 3′)” column. More specifically, D1-D9 are bulged duplexes that each include a bulged cytidine located between adenosine moieties; D10-D13 are bulged duplexes that each include a sliding bulge from adjacent adenosine moieties (i.e., a bulged adenosine between adjacent adenosine moieties); D14-D17 are bulged duplexes that each include a bulged cytidine between adjacent guanosine moieties; D18-D21 are bulged duplexes that each include a bulged thymidine between adjacent guanosine moieties; D22-D25 are bulged duplexes that each include a bulged adenosine between adjacent guanosine moieties; and D26-D29 are bulged duplexes that each include a sliding bulge from adjacent guanosine moieties (i.e., a bulged guanosine between adjacent guanosine moieties).

The data shown in the “Discrimination” and “Increase of Discrimination” columns demonstrates the impact on discrimination of the various LNA motifs for each type of bulge. Table 1 includes oligonucleotide probes having multiple continuous LNA residues and oligonucleotide probes having multiple discontinuous LNA residues. “Increase in Discrimination” refers to the increase in discrimination of a given LNA-containing probe as compared to the equivalent unmodified DNA probe, which is the first probe presented for each set or group (i.e., the respective probe of duplexes D1, D10 D14, D18, D22, and D26).

The data in Table 1 demonstrates that the largest increase in discrimination occurred in probes including consecutive sets of three LNA residues (i.e., triplets) centered on the bulged nucleotide for C, T, and G bulges. In particular, the probe of duplex D3 provides the highest discrimination and the highest increase in discrimination of the probes used to generate duplexes D1-D9, the probe of duplex D16 provides the highest discrimination and the highest increase in discrimination of the probes used to generate duplexes D14-D17, the probe of duplex D20 has the highest increase in discrimination of the probes used to generate D18-D21, and the probe of duplex D28 has the highest increase in discrimination of the probes used to generate D26-29. Accordingly, among examined probes including C, T, and G bulges in a bulged duplex, discrimination was maximized by those that included an LNA residue as the bulged residue and LNA residues as both of the two nucleotides adjacent to the LNA residue, such that the bulge is adjacently flanked in each of the 5′ and 3′ directions by one additional LNA residue. Notably, this motif caused the highest increase of discrimination in both static and sliding bulges where the bulge nucleotide is a C, T, or G moiety. The probe sequences for duplexes D1-D29 correspond to SEQ IDs: 1-29, respectively.

The data in Table 1 also indicates that LNA modifications of A bulges have a low and, in some instances, negligible impact on discrimination. Generally speaking, the discrimination measured for D11-D13 and D23-D25 was generally similar. As all LNA patterns examined in Table 1 yielded similar increases in discrimination for A bulges and as will be described in more detail subsequently, design rules for single bulges based on the data of Table 1 for C, T, and G bulges can also be applied to A bulges to create general design rules.

TABLE 1
Effect on melting temperatures and discrimination (°C) of
various patterns of LNA modifications and bulge positions
in oligonucleotide probe sequences.Bulge nucleotides
are underlined and the probe sequences are grouped
according to the underlined headings.

| | Oligonucleotide | | | Reference | | Increase of |
|---|---|---|---|---|---|---|
| Bulged | Probe Sequence | | Bulged | Matched | Discrimi- | Discrimi- |
| Duplex | (5′ LNA | | Duplex | Duplex | nation | nation |
| ID | to 3′) | Count | Tₘ (°C) | Tₘ (°C) | ΔTₘ (ºC) | (°C) |
| | C bulge in AA/TT | | | | | |
| D1 | AAGTCCACAGTGATC | 0 | 49.0 | 61.6 | −12.6 | 0.0 |
| D2 | AAGTCCA+CAGTGATC | 1 | 49.7 | 65.4 | −15.7 | 3.1 |
| D3 | AAGTCC+A+C+AGTGATC | 3 | 49.4 | 70.0 | −20.6 | 8.0 |
| D4 | AAGTCC+AC+AGTGATC | 2 | 50.4 | 67.0 | −16.6 | 4.0 |
| D5 | AAGTC+CACA+GTGATC | 2 | 52.2 | 66.9 | −14.7 | 2.1 |
| D6 | AAG+TCCACAGT+GATC | 2 | 47.8 | 65.0 | −17.2 | 4.6 |
| D7 | AAGTC+CA+CA+GTGATC | 3 | 52.6 | 72.0 | −19.4 | 6.8 |
| D8 | AAGTC+CACA+GT+GATC | 3 | 55.8 | 71. | −15.3 | 2.7 |
| D9 | AAG+TC+CACA+GT+GATC | 4 | 54.6 | 73.0 | −18.4 | 5.8 |
| | A bulge in AA/TT | | | | | |
| | (Sliding Bulge) | | | | | |
| D10 | AAGTCCAAAGTGATC | 0 | 50.3 | 59.9 | −9.6 | 0.0 |
| D11 | AAGTCCA+AAGTGATC | 1 | 51.4 | 61.8 | −10.4 | 0.8 |
| D12 | AAGTCC+A+A+AGTGATC | 3 | 55.4 | 65.5 | −10.1 | 0.5 |
| D13 | AAGTCC+AA+AGTGATC | 2 | 53.8 | 63.9 | −10.1 | 0.5 |
| | C bulge in GG/CC | | | | | |
| D14 | GAGAGCGACCAC | 0 | 40.9 | 60.1 | −19.2 | 0.0 |
| D15 | GAGAG+CGACCAC | 1 | 41.9 | 64.0 | −22.1 | 2.9 |
| D16 | GAGA+G+C+GACCAC | 3 | 43.8 | 70.8 | −27.0 | 7.8 |
| D17 | GAGA+GC+GACCAC | 2 | 43.8 | 65.9 | −22.1 | 2.9 |
| | T bulge in GG/CC | | | | | |
| D18 | GAGAGTGACCAC | 0 | 40.7 | 54.7 | −14.0 | 0.0 |
| D19 | GAGAG+TGACCAC | 1 | 42.8 | 58.4 | −15.6 | 1.6 |
| D20 | GAGA+G+T+GACCAC | 3 | 44.7 | 66.8 | −22.1 | 8.1 |
| D21 | GAGA+GT+GACCAC | 2 | 43.5 | 62.1 | −18.6 | 4.6 |
| | A bulge in GG/CC | | | | | |
| D22 | GAGAGAGACCAC | 0 | 41.2 | 54.1 | −12.9 | 0.0 |
| D23 | GAGAG+AGACCAC | 1 | 45.7 | 57.9 | −12.2 | -0.7 |
| D24 | GAGA+G+A+GACCAC | 3 | 54.7 | 66.4 | −11.7 | -1.2 |
| D25 | GAGA+GA+GACCAC | 2 | 50.5 | 62.5 | −12.0 | -0.9 |
| | G bulge in GG/CC | | | | | |
| | (Sliding Bulge) | | | | | |
| D26 | GAGAGGGACCAC | 0 | 43.7 | 56.5 | −12.8 | 0.0 |
| D27 | GAGAG+GGACCAC | 1 | 47.7 | 62.1 | −14.4 | 1.6 |
| D28 | GAGA+G+G+GACCAC | 3 | 58.4 | 74.4 | −16.0 | 3.2 |
| D29 | GAGA+GG+GACCAC | 2 | 53.4 | 68.8 | −15.4 | 2.6 |

Table 2 below presents melting temperature data that demonstrates the effect on oligonucleotide probe discrimination of the relative positioning of LNA residues and multi-nucleotide bulges within oligonucleotide probes. Table 2 presents the reference-matched duplex melting temperature as well as bulged duplex melting temperature for various LNA-containing nucleotide probes and various bulge positions. The relative position of the bulge (i.e., in the bulged duplex) and the LNA(s) were varied to understand the impact of LNA quantity, LNA position, and bulge position on oligonucleotide discrimination.

As is described in the caption for Table 2, the location of each bulge nucleotide in the bulged duplex is indicated by an underline. The oligonucleotide probe sequences presented in Table 2 are generally grouped according to the number of bulge nucleotides examined. More specifically, D30-D33 are bulged duplexes that each include two bulged cytidine residues; D34-D36 are bulged duplexes that each include three bulged cytidine residues; and D37-D41 are bulged duplexes that each include four bulged cytidine residues. C bulges were considered to be sufficiently representative of other bulge nucleotide identities based on the similar impacts that LNA position had on discrimination for C, T, and G residues according to the data of Table 1. “Increase in Discrimination” refers to the increase in discrimination of a given LNA-containing probe as compared to the equivalent unmodified DNA probe, which is the first probe presented for each set or group (i.e., the respective probe of probes D30, D34, and D37). The probe sequences for duplexes D30-D41 are SEQ IDs: 30-41, respectively.

For oligonucleotides containing a two-cytidine bulge (i.e., in the bulge duplex), the highest discrimination was observed for the probe of D32, in which both bulge nucleotides were LNA residues and each adjacent nucleotide was also an LNA residue (i.e., the 5′ and 3′ nucleotides that adjacently flank the bulge nucleotides were both LNA residues). For oligonucleotides containing a three-cytidine bulge, the highest discrimination was observed for the probe of D35, in which the three bulge nucleotides were LNA residues and no other residues were LNA residues (i.e., only the three bulge nucleotides were LNA residues). Notably, however, the probe of D36 also showed significantly increased discrimination. D36 included LNA residues at each nucleotide flanking the bulge nucleotides (i.e., at both the 5′ and 3′ nucleotides that adjacently flank the bulge nucleotides) and at the 5′ and 3′ ends of the bulge region. For oligonucleotides containing a four-nucleotide bulge, the highest discrimination was observed for the probe of D38, in which the four bulge nucleotides were LNA residues and no other residues were LNA residues (i.e., only the four bulge nucleotides were LNA residues). However, the probe of D40 also showed a significant increase in discrimination. The probe of D40 included LNA residues at each nucleotide flanking the bulge nucleotides (i.e., at both the 5′ and 3′ nucleotides that adjacently flank the bulge nucleotides) and at the center two bulge nucleotides, such that the 5′ and 3′ ends of the bulge region were unmodified nucleotides. Notably, regardless of bulge size, all probes modified with LNA residues shown in Table 2 exhibited a significant increase in discrimination (more than 3 °C) as compared to corresponding unmodified DNA probes.

TABLE 2
Effect on melting temperatures and discrimination
(C) of various patterns of LNA modifications
and multi-nucleotide bulges in probe sequences.
Bulged nucleotides in the probe sequence are underlined.

| | | | | | Reference | | Increase of |
|---|---|---|---|---|---|---|---|
| Bulged | Oligonucleotide | | | Bulged | Matched | Discrimi- | Discrimi- |
| Duplex | Probe Sequence | LNA | Bulge | Duplex | Duplex | nation | nation |
| ID | (5′ to 3′) | Count | size | Tₘ (°C) | Tₘ (°C) | ΔTₘ (ºC) | (°C) |
| D30 | AAGTCCACCAGTGATC | 0 | 2 | 42.8 | 65.0 | −22.2 | 0.0 |
| D31 | AAGTCCA+C+CAGTGATC | 2 | 2 | 41.2 | 72.1 | −30.9 | 8.7 |
| D32 | AAGTCC+A+C+C+AGTGATC | 4 | 2 | 43.2 | 77.0 | −33.8 | 11.6 |
| D33 | AAGTCC+ACC+AGTGATC | 2 | 2 | 44.2 | 69.7 | −25.5 | 3.3 |
| D34 | AAGTCCACCCAGTGATC | 0 | 3 | 38.1 | 67.5 | −29.4 | 0.0 |
| D35 | AAGTCCA+C+C+CAGTGATC | 3 | 3 | 38.2 | 78.2 | −40.0 | 10.6 |
| D36 | AAGTCC+A+CC+C+AGTGATC | 4 | 3 | 41.2 | 80.6 | −39.4 | 10.0 |
| D37 | AAGTCCACCCCAGTGATC | 0 | 4 | 33.2 | 70.1 | −36.9 | 0.0 |
| D38 | AAGTCCA+C+C+C+CAGTGATC | 4 | 4 | 36.3 | 84.2 | −47.9 | 11.0 |
| D39 | AAGTCC+A+CCC+C+AGTGATC | 4 | 4 | 41.1 | 82.3 | −41.2 | 4.3 |
| D40 | AAGTCC+AC+C+CC+AGTGATC | 4 | 4 | 37.0 | 84.1 | −47.1 | 10.2 |
| D41 | AAGTCCAC+C+CCAGTGATC | 2 | 4 | 35.8 | 76.4 | −40.6 | 3.7 |

Table 3 below presents melting temperature data that demonstrates the effect on oligonucleotide probe discrimination of the relative positioning of LNA residues and single nucleotide bulges in the target sequence in the bulged duplex. That is, for the data presented in Table 3, the bulge in the bulged duplex was located in the strand complementary to the probe oligonucleotide. Table 3 presents the reference-matched duplex melting temperature as well as bulged duplex melting temperature for various LNA-containing nucleotide probes and various target sequence bulge positions. The relative position of the bulge (i.e., in the bulged duplex) and the LNA(s) were varied to understand the impact of LNA quantity, LNA position, and bulge position on oligonucleotide discrimination.

As indicated in the caption for Table 3, the location of the bulge nucleotide in the opposite, target strand in the bulged duplex is indicated by an underline. The target sequence used to collect the data in Table 3 was 3′-CACCAGXGAGAG-5′, where X is the bulged residue in the bulged duplex and where X is omitted from the target sequence in the reference-matched duplex. The oligonucleotide probe sequences presented in Table 3 are grouped according to the type of bulge being examined, which is indicated by the underlined headings in the “Oligonucleotide Probe Sequence (5′ to 3′)” column. More specifically, the data for D42-D45 examine various bulge duplexes where the target strand includes a bulged cytidine; the data for D46-D49 examine various bulge duplexes where the target strand includes a bulged thymidine; the data for D50-D53 examine various bulge duplexes where the target strand includes a bulged adenosine; and the data for D54-D57 examine various bulge duplexes where the target strand includes a bulged guanosine. “Increase in Discrimination” refers to the increase in discrimination of a given LNA-containing probe as compared to the equivalent unmodified DNA probe, which is the first probe presented for each set or group (i.e., the respective probe of probes D42, D46, D50, and D54). The probe sequences for duplexes D42-D57 are SEQ IDs: 42-57, respectively. The target sequence for duplexes D42-45 is SEQ ID: 180; the target sequence for duplexes 46-49 is SEQ ID: 181; the target sequence for duplexes 50-53 is SEQ ID: 182; and the target sequence for duplexes 54-57 is SEQ ID: 183.

Oligonucleotide probe sequences for D44, D48, D52, D56 had both the largest discrimination and the largest increase of discrimination for bulged C, T, A, and G residues, respectively. Notably, the probes for each of D44, D48, D52, D56 include four LNAs at the closing base pairs at the bulge site. That is, the probes for each of D44, D48, D52, D56 contain four consecutive LNAs that base pair with residues that flank the bulged residue in the target sequence—two LNA residues that base pair with nucleotides that are directly adjacent to the bulged nucleotide as well as two LNA residues that adjacently flank those aforementioned two LNA residues, such that there are four consecutive LNA residues with two base paired to the two residues on the 5′ end of the bulge and the remaining two base paired to the two residues on the 3′ end of the bulge in the bulged duplex.

TABLE 3
Effect on melting temperatures and discrimination (°C)
of various patterns of LNA modifications of probe
hybridization to target DNA sequences. The target
sequence used was 3′-CACCAGXGAGAG-5′, where X is
one of C, T, A, and G according to the underlined
headings within the table. The underscores (“_”) in
the probe sequences indicate the location on the
probe where the bulged nucleotide is located on
the opposite, target sequence.

| | | | | Reference | | Increase of |
|---|---|---|---|---|---|---|
| Bulged | | | Bulged | Matched | Discrimi- | Discrimi- |
| Duplex | Oligonucleotide Probe | LNA | Duplex | Duplex | nation | nation |
| ID | Sequence (5′ to 3′) | Count | Tₘ (°C) | Tₘ (°C) | ΔTₘ (ºC) | (°C) |
| | X = C | | | | | |
| D42 | GTGGTC_CTCTC | 0 | 40.9 | 51.6 | −10.7 | 0.0 |
| D43 | GTGGT+C+CTCTC | 2 | 49.3 | 60.1 | −10.8 | 0.1 |
| D44 | GTGG+T+C +C+TCTC | 4 | 57.1 | 69.0 | −11.9 | 1.2 |
| D45 | GTGG+TC_C+TCTC | 2 | 49.3 | 60.2 | −10.9 | 0.2 |
| | X = T | | | | | |
| D46 | GTGGTC_CTCTC | 0 | 40.7 | 51.6 | −10.9 | 0.0 |
| D47 | GTGGT+C_+CTCTC | 2 | 48.6 | 60.1 | −11.5 | 0.6 |
| D48 | GTGG+T+C_+C+TCTC | 4 | 56.4 | 69.0 | −12.6 | 1.7 |
| D49 | GTGG+TC_C+TCTC | 2 | 48.6 | 60.2 | −11.6 | 0.7 |
| | X = A | | | | | |
| D50 | GTGGTC_CTCTC | 0 | 41.2 | 51.6 | −10.4 | 0.0 |
| D51 | GTGGT+C_+CTCTC | 2 | 45.4 | 60.1 | −14.7 | 4.3 |
| D52 | GTGG+T+C_+C+TCTC | 4 | 53.3 | 69.0 | −15.7 | 5.3 |
| D53 | GTGG+TC_C+TCTC | 2 | 47.7 | 60.2 | −12.5 | 2.1 |
| | X = G (Sliding Bulge) | | | | | |
| D54 | GTGGTC_CTCTC | 0 | 43.7 | 51.6 | −7.9 | 0.0 |
| D55 | GTGGT+C_+CTCTC | 2 | 50.1 | 60.1 | −10.0 | 2.1 |
| D56 | GTGG+T+C_+C+TCTC | 4 | 58.6 | 69.0 | −10.4 | 2.5 |
| D57 | GTGG+TC_C+TCTC | 2 | 50.0 | 60.2 | −10.2 | 2.3 |

Table 4, below, presents melting temperature data that demonstrates the effect on oligonucleotide probe discrimination of the relative positioning of LNA residues and both single- and multi-nucleotide bulges in the target sequence in the bulged duplex. Like the data presented in Table 3, the data presented in Table 4 describes discrimination where the bulge in the bulged duplex was located in the strand complementary to the probe oligonucleotide. Table 4 presents the reference-matched duplex melting temperature as well as bulged duplex melting temperature for various LNA-containing nucleotide probes and various target sequence bulge positions. The relative position of the bulge (i.e., in the bulged duplex), the size of the bulge (i.e., the number of bulged nucleotides in the bugled duplex), and the LNA(s) were varied to understand the impact of LNA quantity, LNA position, and bulge position on oligonucleotide discrimination.

As indicated in the caption for Table 4, the location of the bulge nucleotide(s) in the opposite, target strand in the bulged duplex is indicated by an underline. The target sequence used to collect the data in Table 4 was 3′-CTAGTGAXACCTGAA-5′, where X is the bulged residue in the bulged duplex and where X is omitted from the target sequence in the reference-matched duplex. The oligonucleotide probe sequences presented in Table 4 are grouped according to the type of bulge being examined, which is indicated by the underlined headings in the “Oligonucleotide Probe Sequence (5′ to 3′)” column. More specifically, the data for D58-D62 examine various bulge duplexes where the target strand includes a bulged guanosine; the data for D63-D67 examine various bulge duplexes where the target strand includes a bulged adenosine; the data for D68-D72 examine various bulge duplexes where the target strand includes a bulged cytidine; the data for D73-D77 examine various bulge duplexes where the target strand includes a two bulged cytidines; the data for D78-D82 examine various bulge duplexes where the target strand includes a three bulged cytidines; the data for D83-D87 examine various bulge duplexes where the target strand includes a four bulged cytidines; and the data for D88-D92 examine various bulge duplexes where the target strand includes a two bulged adenosines as a sliding bulge (i.e., due to the adenosine residues flanking the bulge site). “Increase in Discrimination” refers to the increase in discrimination of a given LNA-containing probe as compared to the equivalent unmodified DNA probe, which is the first probe presented for each set or group (i.e., the respective probe of duplexes D58, D63, D68, D73, D78, D83, and D88).

The data shown in Table 4 is generally supportive of the role of the motif discussed with respect to the data of Table 3, in which LNA residues are substituted at closing base pairs of the bulge. For some bulges, such as G, CC, and CCCC, a closing duplet of LNAs (i.e., where LNAs base pair to each nucleotide flanking the bulge nucleotides) produced the highest discrimination. For other bulges, such as A, CCC, and AA, a closing tetraplet of LNAs (i.e., the motif of four LNAs at the closing site, where two are located on the 5′ end of the bulge and two are located on the 3′ end of the bulge) produced the highest discrimination. Considering that the propagated standard error for increase of discrimination is 0.6 °C, some differences between LNA probes are not statistically significant. For bulges of a single cytidine residue, probes having two or four LNA modifications at the bulge site show similar discrimination that is within this experimental error. Further, a comparison of the data presented in Table 1, Table 2, Table 3, and Table 4 indicates that the increase in discrimination provided by LNA-containing oligonucleotide probes is larger where the bulge is present in the probe (i.e., in a bulged duplex) than in the complementary, target sequence (i.e., in a bulged duplex).

All of the aforementioned motifs produced an increased discrimination for all bulge types and, notably, LNA residues spaced to base pair to target sequence nucleotides three and four nucleotides from the bulge nucleotides generally had no impact or a negative impact on discrimination. In particular, LNAs spaced to base pair away from the bulge site reduced discrimination for G, A, C, CC, CCCC, and AA bulges. The probe sequences for duplexes D58-D92 are SEQ IDs: 58-92, respectively. The target sequence for duplexes D58-62 is given by SEQ ID: 184; the target sequence for duplexes 63-67 is given by SEQ ID: 185; the target sequence for duplexes 68-72 is given by SEQ ID: 186; the target sequence for duplexes 73-77 is given by SEQ ID: 187; the target sequence for duplexes 78-82 is given by SEQ ID: 188; the target sequence for duplexes 83-87 is given by SEQ ID: 189; and the target sequence for duplexes 88-92 is given SEQ ID: 190.

TABLE 4
Effect on melting temperatures and discrimination (°C) of
various patterns of LNA modifications of probe hybridization
to target DNA sequences. The target sequence used was
3′-CTAGTGAXACCTGAA-5′, where the sequence of X is denoted
according to the underlined headings within the table. The
underscores (“_”) in the probe sequences indicate the
location on the probe where the bulged nucleotide is
located on the opposite, target sequence.

| | | | | Reference | | Increase of |
|---|---|---|---|---|---|---|
| Bulged | | | Bulged | Matched | Discrimi- | Discrimi- |
| Duplex | Oligonucleotide Probe | LNA | Duplex | Duplex | nation | nation |
| ID | Sequence (5′ to 3′) | Count | Tₘ (°C) | Tₘ (°C) | ΔTₘ (ºC) | (°C) |
| | X = G | | | | | |
| D58 | GATCACT_TGGACTT | 0 | 47.4 | 57.7 | −10.3 | 0.0 |
| D59 | GATCAC+T_+TGGACTT | 2 | 49.9 | 62.7 | −12.8 | 2.5 |
| D60 | GATCA+C+T_+T+GGACTT | 4 | 60.6 | 72.7 | −12.1 | 1.8 |
| D61 | GATCA+CT_T+GGACTT | 2 | 54.3 | 65.9 | −11.6 | 1.3 |
| D62 | GA+T+CACT_TGGACTT | 2 | 54.7 | 64.2 | −9.5 | −0.8 |
| | X = A (Sliding Bulge) | | | | | |
| D63 | GATCACT_TGGACTT | 0 | 50.3 | 57.7 | −7.4 | 0.0 |
| D64 | GATCAC+T_+TGGACTT | 2 | 53.5 | 62.7 | −9.2 | 1.8 |
| D65 | GATCA+C+T_+T+GGACTT | 4 | 63.2 | 72.7 | −9.5 | 2.1 |
| D66 | GATCA+CT_T+GGACTT | 2 | 57.5 | 65.9 | −8.4 | 1.0 |
| D67 | GA+T+CACT_TGGACTT | 2 | 57.6 | 64.2 | −6.6 | −0.8 |
| | X = C | | | | | |
| D68 | GATCACT_TGGACTT | 0 | 49.0 | 57.7 | −8.7 | 0.0 |
| D69 | GATCAC+T_+TGGACTT | 2 | 52.8 | 62.7 | −9.9 | 1.2 |
| D70 | GATCA+C+T_+T+GGACTT | 4 | 63.0 | 72.7 | −9.7 | 1.0 |
| D71 | GATCA+CT_T+GGACTT | 2 | 55.7 | 65.9 | −10.2 | 1.5 |
| D72 | GA+T+CACT_TGGACTT | 2 | 55.9 | 64.2 | −8.3 | −0.4 |
| | X = CC | | | | | |
| D73 | GATCACT_TGGACTT | 0 | 42.8 | 57.7 | −14.9 | 0.0 |
| D74 | GATCAC+T_+TGGACTT | 2 | 45.8 | 62.7 | −16.9 | 2.0 |
| D75 | GATCA+C+T_+T+GGACTT | 4 | 56.5 | 72.7 | −16.2 | 1.3 |
| D76 | GATCA+CT_T+GGACTT | 2 | 49.3 | 65.9 | −16.6 | 1.7 |
| D77 | GA+T+CACT_TGGACTT | 2 | 49.4 | 64.2 | −14.8 | −0.1 |
| | X = CCC | | | | | |
| D78 | GATCACT_TGGACTT | 0 | 38.1 | 57.7 | −19.6 | 0.0 |
| D79 | GATCAC+T_+TGGACTT | 2 | 41.2 | 62.7 | −21.5 | 1.9 |
| D80 | GATCA+C+T_+T+GGACTT | 4 | 50.8 | 72.7 | −21.9 | 2.3 |
| D81 | GATCA+CT_T+GGACTT | 2 | 44.6 | 65.9 | −21.3 | 1.7 |
| D82 | GA+T+CACT_TGGACTT | 2 | 44.4 | 64.2 | −19.8 | 0.2 |
| | X = CCCC | | | | | |
| D83 | GATCACT_TGGACTT | 0 | 33.2 | 57.7 | −24.5 | 0.0 |
| D84 | GATCAC+T_+TGGACTT | 2 | 36.5 | 62.7 | −26.2 | 1.7 |
| D85 | GATCA+C+T_+T+GGACTT | 4 | 48.0 | 72.7 | −24.7 | 0.2 |
| D86 | GATCA+CT_T+GGACTT | 2 | 41.0 | 65.9 | −24.9 | 0.4 |
| D87 | GA+T+CACT_TGGACTT | 2 | 41.1 | 64.2 | −23.1 | −1.4 |
| | X = AA (Sliding Bulge) | | | | | |
| D88 | GATCACT_TGGACTT | 0 | 46.9 | 57.7 | −10.8 | 0.0 |
| D89 | GATCAC+T_+TGGACTT | 2 | 49.1 | 62.7 | −13.6 | 2.8 |
| D90 | GATCA+C+T_+T+GGACTT | 4 | 55.6 | 72.7 | −17.1 | 6.3 |
| D91 | GATCA+CT T+GGACTT | 2 | 53.3 | 65.9 | −12.6 | 1.8 |
| D92 | GA+T+CACT_TGGACTT | 2 | 54.1 | 64.2 | −10.1 | −0.7 |

Table 5 provides melting temperature data that demonstrates the effect on oligonucleotide probe discrimination of the relative positioning of LNA residues and internal loops of two consecutive mismatched residues. Table 5 presents the reference-matched duplex melting temperature as well as mismatched duplex melting temperature for various LNA-containing nucleotide probes, various mismatched residue positions, and various mismatched residue identities.

As indicated in the caption for Table 5, the location of each mismatched nucleotide in the mismatched duplex is indicated by an underline. The oligonucleotide probe sequences presented in Table 5 are grouped according to the type of mismatched present in the mismatched duplex, which is indicated by the underlined headings in the “Oligonucleotide Probe Sequence (5′ to 3′)” column. More specifically, duplexes D93-D98 include a TT/GG mismatch and duplexes D99-D104 include a TT/CC mismatch. “Increase in Discrimination” refers to the increase in discrimination of a given LNA-containing probe as compared to the equivalent unmodified DNA probe, which is the first probe presented for each set or group (i.e., the respective probe of duplexes D93 and D99). The probe sequences for duplexes 93-104 are given by SEQ IDs: 93-104, respectively.

Increases of discrimination were observed for probes having LNAs on mismatched residues and/or LNAs adjacent to mismatched residues (i.e., in duplexes D94-D96 and D100-D102). The largest increase in discrimination was observed for oligonucleotides including LNA tetraplets centered on the two mismatched nucleotides (i.e., in duplexes D95 and D101). That is, the largest increase in discrimination was observed for oligonucleotides where the two mismatched nucleotides and the two nucleotides adjacently flanking the mismatched nucleotides on the 5′ and 3′ sides of the mismatch are LNA moieties. Further, the data of Table 5 indicates that LNA moieties that are spaced multiple nucleotides away from the mismatch site (i.e., with no intervening LNA moieties) reduce discrimination.

TABLE 5
Effect on melting temperatures and discrimination (ºC)
of various patterns of LNA modifications of probe
hybridization to a target DNA sequence. The underscores
(“_”) in the probe sequences indicate the location of
nucleotide mismatches between the probe and the target sequence.

| | | | | Reference | | Increase of |
|---|---|---|---|---|---|---|
| | Oligonucleotide | | Mismatched | Matched | Discrimi- | Discrimi- |
| Mismatched | Probe Sequence | LNA | Duplex | Duplex | nation | nation |
| Duplex ID | (5′ to 3′) | Count | Tₘ (°C) | Tₘ (°C) | ΔTₘ (ºC) | (°C) |
| | TT/GG mismatches | | | | | |
| D93 | CACAGTGCATTTCA | 0 | 44.2 | 58.3 | −14.1 | 0.0 |
| D94 | CACAGTGCA+T+TTCA | 2 | 46.3 | 61.8 | −15.5 | 1.4 |
| D95 | CACAGTGC+A+T+T+TCA | 4 | 49.2 | 65.7 | −16.5 | 2.4 |
| D96 | CACAGTGC+ATT+TCA | 2 | 45.7 | 62.1 | −16.4 | 2.3 |
| D97 | CACAGTG+CATTT+CA | 2 | 53.2 | 65.8 | −12.6 | −1.5 |
| D98 | CAC+AG+TGCATTTCA | 2 | 52.5 | 65.4 | −12.9 | −1.2 |
| | TT/CC mismatches | | | | | |
| D99 | CACAGTGCATTTCA | 0 | 39.8 | 58.3 | −18.5 | 0.0 |
| D100 | CACAGTGCA+T+TTCA | 2 | 41.6 | 61.8 | −20.2 | 1.7 |
| D101 | CACAGTGC+A+T+T+TCA | 4 | 44.2 | 65.7 | −21.5 | 3.0 |
| D102 | CACAGTGC+ATT+TCA | 2 | 41.4 | 62.1 | −20.7 | 2.2 |
| D103 | CACAGTG+CATTT+CA | 2 | 47.5 | 65.8 | −18.3 | −0.2 |
| D104 | CAC+AG+TGCATTTCA | 2 | 48.5 | 65.4 | −16.9 | −1.6 |

Table 6 provides melting temperature data that demonstrates the effect on oligonucleotide probe discrimination of the relative positioning of LNA residues and internal loops of mismatched residues. Table 6 presents the reference-matched duplex melting temperature as well as mismatched duplex melting temperature for various LNA-containing nucleotide probes, various mismatched residue positions, and various mismatched residue identities. The data of table 6 particularly focuses on A/G mismatches, which are known to form unusually sheared base pairs, as described in Li Y., and Agrawal S., Oligonucleotides containing G.A pairs: Effect of flanking sequences on structure and stability, Biochemistry 34 (1995) 10056-10062. The data of Table 6 examines both consecutive A/G mismatches and single A/G mismatches. Table 6 indicates the type of mismatch in the “Mismatch Type” column and provides the identity of mismatched residues on the probe before the slash and the identity of the mismatched residues on the target after the slash. “GA/AG” refers to consecutive mismatches, and both G/A and A/G refer to a single mismatches. Table 6 provides data for both consecutive and single mismatches. In particular, duplex D105 contains both of the A/G mismatches that are present in single-mismatch duplexes D110 and D112. Similarly, duplex D107 contains both A/G that are present in single-mismatch duplexes D111 and D113).

As indicated in the caption for Table 6, the location of each mismatched nucleotide in the mismatched duplex is indicated by an underline. As with other tables presented herein, “Increase in Discrimination” refers to the increase in discrimination of a given LNA-containing probe as compared to the equivalent unmodified DNA probe, which is the first probe presented for each set or group (i.e., the probes of duplexes D105, D110, and D112). The probe sequences for duplexes D105-D113 are given by SEQ IDs: 105-113, respectively.

For the consecutive mismatch group, increases of discrimination were observed for probes having LNAs on mismatched residues and/or LNAs adjacent to mismatched residues (i.e., in duplexes D106-108). The largest discrimination and increase in discrimination for the consecutive mismatch group was observed for oligonucleotides including LNA tetraplets centered on the two mismatched nucleotides (i.e., in duplex D107). That is, the largest increase in discrimination was observed for oligonucleotides where the two mismatched nucleotides and the two nucleotides adjacently flanking the mismatched nucleotides on the 5′ and 3′ sides of the mismatch are LNA moieties.

Unmodified consecutive mismatched duplex D105 has a melting temperature of 59.8 °C, which is higher than the melting temperatures of the single-mismatch duplexes D110 D112, which include the same probe sequence as duplex D105. Because of the high melting temperature and relative stability of duplex D105, the discrimination measured for duplex D105 was relatively low at −1.9 °C. The introduction of an LNA tetraplet in duplex D107 centered on the two mismatched nucleotides suppressed the stability of duplex 105 and significantly improved discrimination to −27.5 °C. Notably, the consecutive mismatched duplex D107 has lower melting temperature than single mismatched duplexes D111 and D113, which include the same oligonucleotide probe as duplex D107. In single nucleotide mismatch groups (i.e., duplexes D110-D111 and D112-D113), the LNA-containing probes showed higher discrimination than the unmodified, non-LNA-containing DNA probe. Collectively, the results demonstrate that LNA-modified probes advantageously increase discrimination.

TABLE 6
Effect on melting temperatures and discrimination (°C) of
various patterns of LNA modifications of probe hybridization
to a target DNA sequence. The underscores (“_”) in the probe
sequences indicate the location of nucleotide mismatches
between the probe and the target sequence.

| | Oligonucleotide | | | | Reference | | Increase of |
|---|---|---|---|---|---|---|---|
| | Probe | | | Mismatched | Matched | Discrimi- | Discrimi- |
| Mismatched | Sequence | LNA | Mismatch | Duplex | Duplex | nation | nation |
| Duplex ID | (5′ to 3′) | Count | type | Tₘ (°C) | Tₘ (°C) | ΔTₘ (ºC) | (°C) |
| D105 | TCAGCGAGCACG | 0 | GA/AG | 59.8 | 61.7 | -1.9 | 0.0 |
| D106 | TCAGC+G+AGCACG | 2 | GA/AG | 44.4 | 67.2 | -22.8 | 20.9 |
| D107 | TCAG+C+G+A+GCACG | 4 | GA/AG | 49.9 | 77.4 | -27.5 | 25.6 |
| D108 | TCAG+CGA+GCACG | 2 | GA/AG | 49.2 | 68.0 | -18.8 | 16.9 |
| D109 | TCA+GCGAG+CACG | 2 | GA/AG | 65.3 | 67.3 | -2.0 | 0.1 |
| D110 | TCAGCGAGCACG | 0 | G/A | 48.3 | 61.7 | -13.4 | 0.0 |
| D111 | TCAG+C+G+A+GCACG | 4 | G/A | 58.7 | 77.4 | -18.7 | 5.3 |
| D112 | TCAGCGAGCACG | 0 | A/G | 54.5 | 61.7 | -7.2 | 0.0 |
| D113 | TCAG+C+G+A+GCACG | 4 | A/G | 64.8 | 77.4 | -12.6 | 5.4 |

Table 7 provides melting temperature data that demonstrates the effect of buffer solutions on probe discrimination for oligonucleotide probes including LNA moieties in examples where the probe forms a bulged duplex and a mismatched duplex. Table 7 presents only increase of discrimination information, but that information is calculated as in Tables 1-6 (i.e., by determining discrimination for an unmodified and modified oligonucleotides based on the mismatched/bulged duplex and reference duplex melting temperatures, and determining the difference in discrimination between the two).

The type of duplex is provided by the underlined headings of Table 7. The duplexes examined by the data of Table 7 appear elsewhere herein and the data of table 7 only provides information relating to the impact of buffer solution on discrimination. In particular, data for duplex D3 is provided by Table 1, data for duplex D69 and duplex D71 is provided by Table 4, and data for duplex D95 is provided by Table 5. 1M Na⁺ buffer refers to the buffer including 1M Na⁺ and PCR buffer refers to the IDT PCR buffer described earlier herein. As indicated by the data of Table 7, buffer choice had low impact on discrimination in bulged duplexes where the bulge is located in the probe, bulged duplexes where the bulge is located in the target sequence, and in mismatched duplexes.

TABLE 7
Effect of different buffer solutions on melting temperatures and
discrimination (°C) of LNA modified oligonucleotide probes
hybridized to a target DNA sequence. The underscores (“_”)
in the oligonucleotide probe sequence and relevant target
sequences (indicated in the mutation type column) indicate
the location of nucleotide mismatches and bulges in the
probe/target sequence hybrids.

| | | | Increase of | |
|---|---|---|---|---|
| | | | Discrimination (°C) in | |
| Duplex | Oligonucleotide Probe | | 1M Na⁺ | |
| ID | Sequence (5′ to 3′) | Mutation Type | buffer | PCR buffer |
| | Bulge in the LNA probe | | | |
| D3 | AAGTCC+A+C+AGTGATC | +A+C+A/TT, C bulge | 8.0 | 8.2 |
| | Bulge in the DNA target | | | |
| D69 | GATCAC+T+TGGACTT | +T+T/ACA, C bulge | 1.2 | 1.1 |
| D71 | GATCA+CTT+GGACTT | TT/ACA, C bulge | 1.5 | 1.2 |
| D95 | Internal Loops/Mismatches | +T+T/GG | 2.4 | 2.5 |
| | CACAGTGC+A+T+T+TCA | | | |

FIG. 4 a graph of discrimination as a function of location of a bulge or mismatch. The X-axis indicates the number of base pairs extending from the 5′ end or 3′ end of the mismatched or bulged duplex and the Y-axis indicates discrimination (°C). All oligonucleotides examined in the data presented in FIG. 4 are 14mers. Accordingly, the X-axis refers to the short of the distance of the 5′ end or the 3′ end of the bulge or mismatch to the 5′ end or the 3′ end of the oligonucleotide, respectively. The sequences of the oligonucleotide probes used to generate the data of FIG. 4 are given in Table 8 below. The sequences were selected to test mismatches and bulges in the same sequence context. The sequence set was designed by repeatedly moving the 3′ base pair to 5′ end, causing the resultant duplexes to have the same mismatch at various positions from the 5′ and 3′ ends of the duplex. The oligonucleotides in the “Reference Matched Duplex” column were duplexed with a fully complementary oligonucleotide for melting temperature measurement. The oligonucleotides listed in the “Inserted C Bulge,” “Inserted CC Bulge,” “T-C Mismatch,” and “T-T Tandem Mismatches” columns included the indicated bulge and/or mismatch in duplex with the strand complementary to the reference-matched duplex. Melting temperatures of these duplexes were experimentally measured. The melting temperatures of the various bulged and mismatched duplexes were compared to the melting temperatures of the reference matched duplex to generate the discrimination data plotted in FIG. 4. As is shown by the data presented in FIG. 4, discrimination improves significantly (i.e., the magnitude increases significantly) when the 5′ end and 3′ end of the bulge or mismatch are spaced by at least three nucleotides from the 5′ end and 3′ end, respectively. Vertical lines 402 and 404 indicate the range of nucleotide positions (i.e., as relative to the 5′ and 3′ ends of the oligonucleotides) that yielded the greatest discrimination for the oligonucleotides of Table 8. The sequences in the “Reference matched duplex,” “T-C Mismatch,” and “TT Tandem Mismatches” columns are given by SEQ IDs: 114-127; the sequences in the “Inserted C Bulge” column are given by SEQ IDs: 128-140; and the sequences in the “Inserted CC Bulge” column are given by SEQ IDs: 141-153.

TABLE 8
List of oligonucleotide sequences used to produce discrimination
data presented in FIG. 4.

| Reference matched | | | | T-T Tandem |
|---|---|---|---|---|
| duplex | Inserted C Bulge | Inserted CC Bulge | T-C Mismatch | Mismatches |
| TTGACTACTCCCAT | TCTGACTACTCCCAT | TCCTGACTACTCCCAT | TTGACTACTCCCAT | TTGACTACTCCCAT |
| TTTGACTACTCCCA | TTCTGACTACTCCCA | TTCCTGACTACTCCCA | TTTGACTACTCCCA | TTTGACTACTCCCA |
| ATTTGACTACTCCC | ATTCTGACTACTCCC | ATTCCTGACTACTCCC | ATTTGACTACTCCC | ATTTGACTACTCCC |
| CATTTGACTACTCC | CATTCTGACTACTCC | CATTCCTGACTACTCC | CATTTGACTACTCC | CATTTGACTACTCC |
| CCATTTGACTACTC | CCATTCTGACTACTC | CCATTCCTGACTACTC | CCATTTGACTACTC | CCATTTGACTACTC |
| CCCATTTGACTACT | CCCATTCTGACTACT | CCCATTCCTGACTACT | CCCATTTGACTACT | CCCATTTGACTACT |
| TCCCATTTGACTAC | TCCCATTCTGACTAC | TCCCATTCCTGACTAC | TCCCATTTGACTAC | TCCCATTTGACTAC |
| CTCCCATTTGACTA | CTCCCATTCTGACTA | CTCCCATTCCTGACTA | CTCCCATTTGACTA | CTCCCATTTGACTA |
| ACTCCCATTTGACT | ACTCCCATTCTGACT | ACTCCCATTCCTGACT | ACTCCCATTTGACT | ACTCCCATTTGACT |
| TACTCCCATTTGAC | TACTCCCATTCTGAC | TACTCCCATTCCTGAC | TACTCCCATTTGAC | TACTCCCATTTGAC |
| CTACTCCCATTTGA | CTACTCCCATTCTGA | CTACTCCCATTCCTGA | CTACTCCCATTTGA | CTACTCCCATTTGA |
| ACTACTCCCATTTG | ACTACTCCCATTCTG | ACTACTCCCATTCCTG | ACTACTCCCATTTG | ACTACTCCCATTTG |
| GACTACTCCCATTT | GACTACTCCCATTCT | GACTACTCCCATTCCT | GACTACTCCCATTT | GACTACTCCCATTT |
| TGACTACTCCCATT | no design | no design | TGACTACTCCCATT | no design |

### Design of LNA-Containing Oligonucleotide Probes

The data provided in Tables 1-7 and FIG. 4 can be used to improve the design and synthesis of LNA-containing probes to improve and/or maximize discrimination.

For oligonucleotides used to detect a mutant allele including an insertion relative to a wildtype allele, the oligonucleotide probe will include a bulge when duplexed with the wildtype allele. The data from Tables 1-2 suggest the following designs to increase and/or maximize discrimination. Where the bulge includes a single nucleotide (e.g., where the mutant allele includes a single base insertion), a triplet of consecutive LNAs including the bulge and the nearest two closing base pairs on the 5′ and 3′ end of the bulge (i.e., the two bases adjacently flanking the bulged base) improves and, in at least some examples, maximizes discrimination. Where the bulge includes two nucleotides (e.g., where the mutant allele includes a two base insertion), a tetraplet of consecutive LNAs including the bulge nucleotides and the nearest two closing base pairs on the 5′ and 3′ end of the bulge (i.e., the two bases adjacently flanking the two bulged bases) improves and, in at least some examples, maximizes discrimination. Where the bulge includes three bases or more (e.g., where mutant allele includes an insertion of three bases or more), consecutive LNAs placed within the bulge increase discrimination more than LNAs placed within closing base pairs (i.e., base pairs adjacent to the bulge on the 5′ or 3′ end). Where the bulge includes more than four nucleotides (e.g., where the mutant allele includes an insertion of four or more bases), the bulge can include an unmodified, native (i.e., non-LNA) nucleotide to limit the size of consecutive LNA modifications, as synthesis of more than four consecutive LNA moieties can be difficult perform.

The aforementioned examples generally refer to oligonucleotide probes for detecting mutants having an insertion relative to the wildtype. In these examples, the probe would be fully complementary to the mutant allele and would include a portion corresponding to the inserted bases (i.e., the bulged residues) that is not complementary to the wildtype allele. However, the above-articulated oligonucleotide design principles can be used to design oligonucleotide probes for other suitable examples where the probe includes a larger number of residues but is at least partially complementary to another, non-reference matched sequence. For example, the above designs can also be used to improve discrimination for oligonucleotides used to detect deletion-insertion mutants where the insertion is one, two, or three or more nucleotides greater in length, respectively, than the deletion. The above designs are yet further applicable to oligonucleotides used to detect a segment of a wildtype genomic sequence in a mixed sample including both the wildtype and a mutant allele including a deletion (i.e., such that the probe would preferentially bind to the wildtype sequence and would form a bulge when duplexed to the mutant allele).

For oligonucleotides used to detect a mutant allele including a deletion relative to a wildtype allele, the wildtype allele will include a bulge when duplexed with the probe. To improve and/or maximize discrimination in these examples, the data of Tables 3-4 suggest including four LNA modifications in the probe at the bulge site. In particular, two LNA moieties at each end of the bulge (i.e., at the nucleotides that base pair with the two nucleotides adjacent to the 5′ end of the bulge and the two nucleotides at the 3′ end of the bulge) can improve and/or maximize discrimination. In some examples, a single LNA moiety at each end of the bulge such that only the closing base pairs opposite the bulge are modified (i.e., such that the nucleotides that base pair with the nucleotide adjacent to the 5′ end of the bulge and the nucleotide adjacent to the 3′ end of the bulge) can significantly improve and/or maximize discrimination. The effect on discrimination of the placement of LNAs within the probe is not significantly affected by the size of the bulge in the complementary strand.

The aforementioned examples generally refer to oligonucleotide probes for detecting mutants having deletion relative to the wildtype. In these examples, the probe would be fully complementary to the mutant allele but would lack bases for base pairing to the nucleotides in the wildtype allele corresponding to the deletion, such that the wildtype allele includes one or more nucleotides that are not complementary to the wildtype allele. However, the above-articulated principles for designing oligonucleotide probe for detecting deletion mutants can be used to design oligonucleotide probes for other suitable examples where the probe includes a smaller number of residues than but is at least partially complementary to another, non-reference matched sequence. For example, the LNA positions for improving specificity (i.e., discrimination) of a probe for detecting a deletion mutant can also be used to improve discrimination for oligonucleotides used to detect deletion-insertion mutants where the insertion includes fewer nucleotides in length than the deletion. The above designs are yet further applicable to oligonucleotides used to detect a segment of a wildtype genomic sequence in a mixed sample including both the wildtype and a mutant allele including an insertion (i.e., such that the probe would preferentially bind to the wildtype sequence and the mutant allele would form a bulge when duplexed to the probe).

LNA-containing oligonucleotide probes can also be used to detect mutant alleles including substitutions relative to corresponding wildtype alleles. Where the mutant allele includes two or more consecutive substitutions relative to the wildtype allele, the duplex of the probe and the wildtype allele forms an internal loop at the mismatched residues. The data of Tables 5-6 can be used to design oligonucleotide probes for detecting mutant alleles including substitutions that improve and/or maximize discrimination. In particular, LNAs at the closing base pairs nearest the internal loop (i.e., adjacent to the 5′ and 3′ ends of the mismatched region) and two LNA nucleotides within the mismatched region (i.e., both nucleotides of an internal loop having two consecutive mismatches). For internal loops of two nucleotides, a tetraplet of LNA moieties including the two mismatched nucleotides and the closing base pairs (i.e., the two nucleotides adjacently flanking the mismatch region) can be used to improve and/or maximize discrimination.

According to the data presented in FIG. 4, locating a bulge (in either the probe or the complementary strand) or mismatch towards the interior of the bulged or mismatched duplex, respectively, increases discrimination by destabilizing the formation of the bulged or mismatched duplex. In particular and per the data presented in FIG. 4, designing oligonucleotide probes such the bulge (in either the probe or the complementary strand) or mismatch at least three nucleotides from either terminus of the probe-target duplex can significantly improve discrimination.

The data presented on FIG. 4 also shows that it is not necessary to locate the bulge or mismatch in the exact center of the probe to improve and/or maximize discrimination. Notably, duplex designs where a bulge or mismatch is off center but at least three nucleotides from a 5′ or 3′ duplex terminus (e.g., located 4, 5, or 6 base pairs from the 5′ or 3′ end) show a similar significant improvement to discrimination as bulges and mismatches positioned in the center of the duplex.

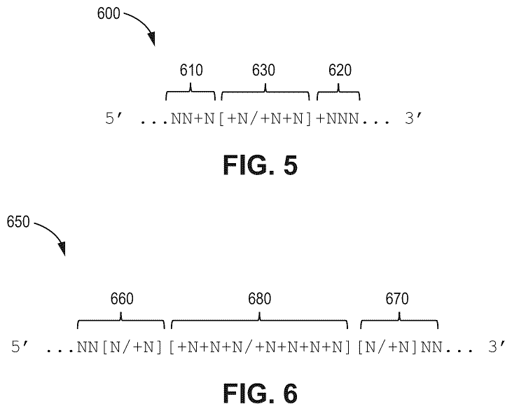

More generally, the design principles articulated based on the collective data of Tables 1-7 can be condensed to several designs for detecting mutant alleles presented in FIG. 5 and FIG. 6. In FIGS. 5 and 6, LNAs are indicated in the oligonucleotide probe sequences by a “plus” (“+”) symbol prior to the nucleotide identity. Further, as FIGS. 5 and 6 are schematic representations meant to facilitate discussion of design principles for oligonucleotide probes, all nucleotide identities are variable and accordingly are indicated by the letter “N.” In FIGS. 5 and 6, ellipses (“ . . . ”) indicate that additional nucleotides may be present, as discussed in more detail subsequently.

FIG. 5 is a schematic depiction of probe 600, which is an LNA-containing oligonucleotide probe. The design principles embodied by probe 600 can be used to detect insertions of one or two nucleotides, various deletion-insertion mutations, substitutions of one or two nucleotides, and deletions of any suitable length. Probe 600 includes fully complementary regions 610 and 620, which are designed to be fully complementary to both the mutant allele and the wildtype allele. Fully complementary regions 610 and 620 adjacently flank semi-complementary region 630 (i.e., are adjacent to the 5′ and 3′ ends, respectively, of semi-complementary region 630). Semi-complementary region 630 includes one or two nucleotides that are fully complementary to the mutant allele and that are not fully complementary to the wildtype allele. Where the mutant allele includes an insertion of one or two nucleotides, the bases of semi-complementary region 630 are complementary to the inserted bases and form a bulge when the probe is duplexed to the wildtype allele. Where the mutant allele includes a substitution of one or two nucleotides, the bases of semi-complementary region 630 are selected to match the sequence of the mutant allele, such that semi-complementary region 630 forms a mismatch region when the probe is duplexed to the wildtype allele. Where the mutant allele includes a deletion, semi-complementary region 630 includes two bases that are complementary to the bases flanking the 5′ and 3′ ends of the deleted nucleotides, such that semi-complementary region 630 is complementary to the closing base pairs of a bulge formed in a duplex with the wildtype sequence.

Where the mutant allele includes a deletion-insertion, the identity of bases of the semi-complementary region can be determined according to whether the deletion-insertion results in fewer nucleotides than the wildtype or the deletion-insertion results in a net gain of nucleotides as compared to the wildtype. Where there is a net loss of nucleotides, the oligonucleotide probes can be designed substantially similarly to the probes described above for detecting a deletion. Where there is a net gain of nucleotides, the oligonucleotide probes can be designed substantially similarly to the probes described above for detecting an insertion.

As depicted in FIG. 5, the nucleotides of semi-complementary region 630 are LNAs, and the nucleotides of fully complementary regions 610 and 620 that are adjacent to semi-complementary region 630 (i.e., that are attached to the 5′ and 3′ ends of semi-complementary region 630) are LNAs. All other nucleotides of the probe can be unmodified nucleotides. Per the improved discrimination from placing a bulged or mismatched region in the interior of the bulged or mismatched duplex, each of fully complementary regions 610 and 620 are at least three nucleotides long.

FIG. 6 is a schematic depiction of probe 650, which is an example of another LNA-containing oligonucleotide probe. Probe 650 can be used to detect insertions of three or four nucleotides and includes fully complementary regions 660 and 670, and further includes semi-complementary region 680. Fully complementary regions 660 and 670 are fully complementary to both the mutant allele and the wildtype allele. Semi-complementary region 680 contains three or four nucleotides that are fully complementary to the three or four inserted nucleotides, respectively. All nucleotides of semi-complementary region 680 are LNA moieties. In some examples, fully complementary regions 660 and 670 only include unmodified (i.e., non-LNA) bases. However, in some examples where semi-complementary region 680 includes only three nucleotides, either fully complementary region 660 or fully complementary region 670 can include a single LNA moiety adjacent to semi-complementary region 680 (i.e., attached to either the 5′ or 3′ end of semi-complementary region 680), such that probe 650 includes four LNA moieties. The probe design of FIG. 6 can also be used to detect deletion-insertion mutations that result in a net gain of three or four consecutive nucleotides.

A particular allele of a biallelic or multiallelic genomic region that is targeted for detection by a probe designed according to the present disclosure can be referred to as a “target allele.” The probe designs of FIG. 5 and FIG. 6 are generally described herein with respect to detecting mutant alleles (as compared to wildtype alleles) for explanatory convenience, but, more generally, the probe designs of FIG. 5 and FIG. 6 can be adapted to detect any specific (i.e., target) allele of a biallelic or multiallelic genomic region, especially where the alleles differ by one or more insertion, deletion, deletion-insertion, and/or substitution mutations based on the structures formed between the oligonucleotide probe and the non-target allele. As a specific example, the probe designs of FIG. 5 and FIG. 6 can be adapted to detect wildtype alleles in lieu of mutant alleles.

The aforementioned LNA-containing probe designs can be used to detect mutations in any suitable genetic material. For example, the oligonucleotide probe designs described herein can be used for oligonucleotide probes for detecting mutations in coding regions of genes, noncoding regions of genes, extragenic regions of a genome, any other suitable region of a genome, and/or any suitable mixture of the foregoing (e.g., a region that overlaps coding and noncoding regions, etc.).

The LNA-containing probes according to the present disclosure can optionally include further modifications. For example, an LNA-containing probe according to the present disclosure can also include a fluorophore and a quencher moiety such that the oligonucleotide probe can be used to perform a 5′ nuclease assay for detecting a target allele, as described subsequently with respect to FIG. 9 and FIG. 10. In at least some of these examples, the fluorophore is attached to the 5′ end of the probe and the quencher is attached to the 3′ end of the probe.

The improved discrimination of probes generated according to the design outlined in FIG. 5 and FIG. 6 enables improved sensitivity for detecting a specific allele of a biallelic or multiallelic genomic region. In particular, the difference in melting temperature between the target allele and the non-target allele(s) is improved by the designs of the present disclosure. Moreover, the designs outlined herein are flexible and can be adapted to a detect wide variety of mutations and/or allelic variations, including substitutions, deletions, insertions, deletion-insertions, etc. The melting temperature of duplexes of LNA-containing probes can be determined via a known technique for use in designing experiments for detecting a specific, target allele. The technique can be, for example, the technique outlined in Owczarzy R., You Y., Growth C. L., and Tataurov A. V., Stability and mismatch discrimination of locked nucleic acid-DNA duplexes, Biochemistry 50 (2011) 9352-9367.

### Example 2—Design of MGB-Containing Oligonucleotide Probes

Tables 9-13 present data that demonstrates the impact on probe discrimination (and, accordingly, probe specificity) of MGB moieties. An MGB moiety of an oligonucleotide probe is able to interact non-covalently with the minor groove of a target-probe duplex to stabilize the duplex, thereby increasing the melting temperature of the target-probe duplex.

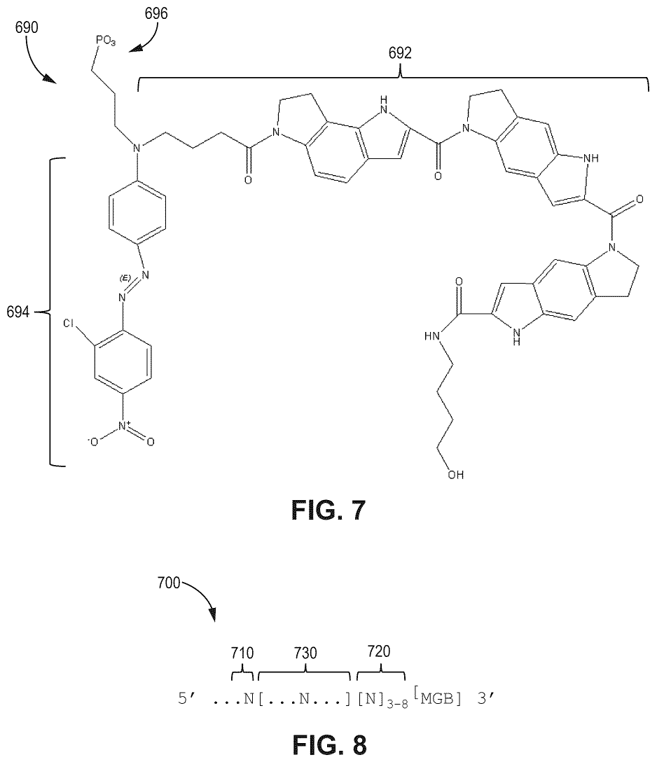

In all examples of oligonucleotide probes including an MGB moiety described herein, the MGB moiety was attached to the 3′ end of the probe sequence. The MGB moiety used in the experiments of Tables 9-13 was 5-(6-(6-(6-(4-N-(3-Dimethoxytrityloxypropyl)-N-(4′-nitro-2′-chloroazobenzen-4-yl)-aminobutanoyl)-3,6,7,8-tetrahydropyrrolo[3,2-e]indole-2-carbonyl)-3,6,7,8-tetrahydropyrrolo[3,2-e]indole-2-carbonyl)-3,6,7,8-tetrahydropyrrolo[3,2-e]indole-2-carboxamido) pentanol, the structure of which is depicted in FIG. 7. FIG. 7 is discussed in more detail subsequently and particular with respect to the description of FIG. 8 However, the oligonucleotide designs based on the data of Tables 9-13 is not specific to any particular MGB moiety and, in other examples, MGB-containing oligonucleotides according to the present disclosure can include a different 3′ MGB moiety. In other examples, any suitable MGB moiety can be used.

Table 9 presents data that demonstrates the effect of the relative positioning of a 3′ MGB moiety and a bulge within the probe (i.e., in a bulged duplex) on discrimination; Table 10 presents data that demonstrates the effect the relative positioning of a 3′ MGB moiety and a bulge in the target sequence (i.e., in a bulged duplex); Tables 11 presents data that demonstrates the effect the relative positioning of a 3′ MGB moiety and one or more mismatch nucleotides in a mismatched duplex; Table 12 examines effect on discrimination of the relative positioning of a 3′ MGB moiety and a bulge within the target sequence (i.e., in a bulged duplex) for bulges of various sizes; and Table 13 examines the role of buffer composition on discrimination for MGB-containing oligonucleotides.

The melting temperature data of Tables 9-13 was generated in substantially the same manner as described herein with respect to the generation of data for Tables 1-7 and FIG. 4. More specifically, the method used by Owczarzy, 2004 was used to collect the data presented in Tables 9-13 using the probes described in the discussions of Tables 9-13 herein. Like the data presented in Tables 1-7 and FIG. 4, the experiments used to generate Tables 9-13 were performed in 1M Na⁺ buffer except for specific experiments presented in the discussion of Table 13, which were performed in IDT PCR buffer.

The data presented in tables 9-13 is the basis of the oligonucleotide probe design strategy for MGB-containing oligonucleotides presented subsequently herein. The discrimination data presented in Tables 1-7 and FIG. 4 is the difference in melting temperature between the reference-matched duplex and one of a bulged duplex and a mismatched duplex, according to the caption of the table and the accompanying description herein. As referred to herein, a “reference-matched” duplex does not contain a bulge, such that the duplex is completely complementary. Conversely, as referred to herein, a “bulged duplex” is only completely complementary at residues other than those indicated as “bulge” nucleotides or “bulged” nucleotides. Moreover, as referred to herein, a “mismatched duplex” is only completely complementary at residues other than those indicated as “mismatched” nucleotides or “mismatch” nucleotides.

Tables 9-12 provide the melting temperature data for MGB-containing oligonucleotide probes as well as melting temperature data for “Native,” non-MGB-containing oligonucleotide probes for each bulge position examined. In each of Tables 9-12, the “ΔTₘ(MGB)” and “ΔTₘ(DNA)” columns provide the difference between the reference-matched duplex (e.g., the “No Bulge” samples listed first in Table 9, Table 10, and the “No Mismatch” sample in Table 11) and the indicated bulged or mismatched duplex. The “Increase of Discrimination” values refer to the increase in discrimination attributed to the presence of MGB and is calculated using the following Equation 1:

Increase of Discrimination=ΔTₘ(DNA)−ΔTₘ(MGB) (Equation 1)

Table 9 below includes melting temperature data and shows the impact of MGB position relative to a bulge nucleotide on probe discrimination. The data presented in Table 9 is specific to the probe oligonucleotide 5′-GTTCAGCTCAGTGT/MGB/(SEQ ID: 153) and was generated by varying the position of a single nucleotide bulge within the probe oligonucleotide (i.e., as opposed to the target sequence). The relative position of the bulge (i.e., in the bulged duplex) was varied to understand the impact of the relative position of the bulge and the MGB moiety. The “Bulge Base” column of Table 9 indicates the position of the bulge nucleotide relative to the MGB moiety, where the nucleotide adjacent to the MGB moiety is assigned the number “1.”

The melting profiles of bulges C8, G9, A10, and C11 using MGB-containing oligonucleotide probes exhibited double transitions and are reported as “DT.” A single, sigmoidal shaped transition is typically expected for melting profiles of oligonucleotide probes and primers (as the temperature of the midpoint of the transition is inferred to be the melting point), meaning that melting data that includes a double transition cannot be used to determine a single melting temperature.

According to the data of Table 9, bulges at G2, A5, and C6 had positive increases in discrimination. That is, bulges separated from the 3′ terminal MGB moiety by one nucleotide, four nucleotides, and five nucleotides had positive changes to discrimination. All other bulge positions either provided a decrease in discrimination or exhibited a double transition. Notably, the bulge at position A5 (separated from the 3′ MGB moiety by four nucleotides) and the bulge at position C6 produced the largest quantity of discrimination, ΔTₘ(MGB) of −16.5 °C and −16.9 °C, respectively. Notably, native oligonucleotide probes exhibited lower values of discrimination and the absolute values of ΔTₘ(DNA) were 15 °C or less for all duplexes. Collectively, the data of Table 9 demonstrates the advantageously improved discrimination oligonucleotide probes modified to include a 3′ MGB moiety and, further, of MGB-containing oligonucleotide probes designed to have a bulge nucleotide located at positions 5 or 6 from a 3′ MGB moiety.

TABLE 9
Effect of various bulge positions within the MGB-containing oligonucleotide
probe 5′-GTTCAGCTCAGTGT/MGB/ on melting temperatures and discrimination.
The underlined text in the oligonucleotide probe sequences indicate the
location of the bulge nucleotide in the probe in various probe/target
sequence hybrids. Melting temperature, discrimination, and increase
of discrimination are in °C.

| | | MGB Oligo- | | Native Oligo- | | |
|---|---|---|---|---|---|---|
| Probe | Bulge | nucleotide | | nucleotide | | Increase of |
| Sequence (5′ to 3′) | Base | Tₘ(MGB) | ΔTₘ(MGB) | Tₘ(DNA) | ΔTₘ(DNA) | Discrimination |
| GTTCAGCTCAGTGT/MGB/ | No | 69.2 | 0.0 | 60.8 | 0.0 | 0.0 |
| | Bulge | | | | | |
| GTTCAGCTCAGTGT/MGB/ | G2 | 64.1 | −5.1 | 57.7 | −3.1 | 2.0 |
| GTTCAGCTCAGTGT/MGB/ | T3 | 63.6 | −5.6 | 54.6 | −6.2 | −0.6 |
| GTTCAGCTCAGTGT/MGB/ | G4 | 59.2 | −10.0 | 49.5 | −11.3 | −1.3 |
| GTTCAGCTCAGTGT/MGB/ | A5 | 52.7 | −16.5 | 51.3 | −9.5 | 7.0 |
| GTTCAGCTCAGTGT/MGB/ | C6 | 52.3 | −16.9 | 45.8 | −15.0 | 1.9 |
| GTTCAGCTCAGTGT/MGB/ | T7 | 60.3 | −8.9 | 48.4 | −12.4 | −3.5 |
| GTTCAGCTCAGTGT/MGB/ | C8 | DT | N/A | 46.3 | −14.5 | N/A |
| GTTCAGCTCAGTGT/MGB/ | G9 | DT | N/A | 44.7 | −16.1 | N/A |
| GTTCAGCTCAGTGT/MGB/ | A10 | DT | N/A | 50.2 | −10.6 | N/A |
| GTTCAGCTCAGTGT/MGB/ | C11 | DT | N/A | 47.9 | −12.9 | N/A |

Table 10 below includes melting temperature data that shows the impact of MGB position relative to a bulge nucleotide on probe discrimination for the probe 5′-TCAGTGATTATTAT/MGB/(SEQ ID: 155). The data of Table 10 was generated by varying the position of the bulge in the target sequence (i.e., in the bulged duplex) rather than the probe sequence, as was discussed with respect to Table 9. Table 10 also presents the reference-matched duplex melting temperature as well as bulged duplex melting temperature for various bulge positions of an MGB-containing oligonucleotide probes. The “Bulge Base” column indicates the position of the bulged base in the target sequence (i.e., that is present in the bulged duplex) relative to the 5′ end of the target sequence. The target sequences of Table 10 are given by SEQ IDs: 156-169.

As shown in Table 10, bulges at C5, C6, C7, C8, and G9 had the largest positive increases in discrimination that exceeded experimental error. As, in a bulged duplex, C5 is located between the fourth and fifth nucleotides of the probe (measured from the 3′ end of the probe sequence) and G9 is located between the eighth and ninth nucleotides of the probe (measured from the 3′ end of the probe sequence), positioning the bulge such that the closing nucleotides in the probe are between the fourth and ninth nucleotide from the 3′ end of the probe provides the positive change (i.e., an increase) in discrimination. Further, positioning the bulge such that the closing nucleotides in the probe are the seventh and eighth nucleotides from the 3′ end (such that the closing nucleotides are spaced from the 3′ end by six and seven nucleotides, respectively) provided the single largest increase in discrimination.

TABLE 10
Effect of bulge positions in target sequences hybridized to
the MGB-containing oligonucleotide probe 5′- TCAGTGATTATTAT/
MGB/ on melting temperatures and discrimination. The
underlined text in the oligonucleotide target sequences
indicate the location of the bulge nucleotide in the target
sequence in various probe- target sequence duplexes.
Melting temperature, discrimination, and increase of
discrimination are in °C.

| Target sequence | Bulge | MGB Oligonucleotide | | Native Oligonucleotide | | Increase of |
|---|---|---|---|---|---|---|
| (3′ to 5′) | Base | Tₘ(MGB) | ΔTₘ(MGB) | Tₘ(DNA) | ΔTₘ(DNA) | Discrimination |
| AGTCACTAATAATA | No Bulge | 71.6 | 0.0 | 48.6 | 0.0 | 0.0 |
| AGTCACTAATAATCA | C2 | 70.8 | −0.8 | 47.7 | −0.9 | −0.1 |
| AGTCACTAATAACTA | C3 | 70.5 | −1.1 | 45.5 | −3.1 | −2.0 |
| AGTCACTAATACATA | C4 | 66.8 | −4.8 | 43.5 | −5.1 | −0.3 |
| AGTCACTAATCAATA | C5 | 60.8 | −10.8 | 39.2 | −9.4 | 1.4 |
| AGTCACTAACTAATA | C6 | 59.8 | −11.8 | 38.1 | −10.5 | 1.3 |
| AGTCACTACATAATA | C7 | 58.2 | −13.4 | 39.8 | −8.8 | 4.6 |
| AGTCACTCAATAATA | C8 | 55.4 | −16.2 | 38.4 | −10.2 | 6.0 |
| AGTCACGTAATAATA | G9 | 57.0 | −14.6 | 36.6 | −12.0 | 2.6 |
| AGTCATCTAATAATA | T10 | 60.8 | −10.8 | 37.6 | −11.0 | −0.2 |
| AGTCTACTAATAATA | T11 | 63.5 | −8.1 | 40.9 | −7.7 | 0.4 |
| AGTACACTAATAATA | A12 | 67.5 | −4.1 | 40.7 | −7.9 | −3.8 |
| AGCTCACTAATAATA | C13 | 69.2 | −2.4 | 43.0 | −5.6 | −3.2 |
| ACGTCACTAATAATA | C14 | 71.9 | 0.3 | 48.2 | −0.4 | −0.7 |

Table 11 below includes melting temperature data and shows the impact of MGB position relative to a mismatched nucleotide on probe discrimination for the probe 5′-TCAGTGATTATTAT/MGB/(SEQ ID: 155). The data of Table 11 was generated by varying the position of the mismatch in the mismatched duplex (i.e., by varying corresponding nucleotide identities of the target sequence). Table 11 also presents the reference-matched duplex melting temperature as well as mismatched duplex melting temperatures for various mismatch positions of an MGB-containing oligonucleotide probes. The “Mismatch Position” column indicates the identity of the mismatch (in a probe-target format) as well as the position of the mismatched base in the probe sequence relative to the 3′ end of the probe.

As shown in Table 11, the addition of an MGB moiety increased mismatch discrimination where mismatch was located in a range of 3 to 8 bases from the MGB moiety. Notably, Tₘ values decreased by more than 10 °C for mismatches located from 4 to 7 nucleotides away from the MGB moiety. Tₘ decreased by 5.9 °C for the mismatch located 3 nucleotides from the MGB moiety (a 0.7 °C increase in discrimination) and Tₘ decreased by 7.2 °C for the mismatch located 8 nucleotides from the MGB moiety (a 1.7 °C increase in discrimination). The highest increases in discrimination were observed where the mismatch was located 5 nucleotides or 6 nucleotides from the MGB moiety (i.e., spaced from the MGB moiety by 4 or 5 nucleotides, respectively). A 5.6 °C increase in discrimination was observed when the mismatch was located 5 nucleotides from the MGB moiety and a 6.4 °C increase in discrimination was observed when the mismatch was located 6 nucleotides from the MGB moiety. The increase in discrimination was, by contrast, 1.2 °C and 2.1 °C when the mismatch was located 4 and 7 nucleotides, respectively from the MGB, moiety.

TABLE 11
Melting temperatures and discrimination of various mismatches between the MGB-containing
oligonucleotide probe 5′-TCAGTGATTATTAT/MGB/ and various target sequences. The underlined
text in the oligonucleotide probe sequences indicate the location in the probe of the
mismatched nucleotide. Melting temperature, discrimination, and increase of
discrimination are in °C.

| | | MGB oligo- | | Native oligo- | | |
|---|---|---|---|---|---|---|
| Probe Sequence | Mismatch | nucleotide | | nucleotide | | Increase of |
| (5′ to 3′) | Position | Tₘ(MGB) | ΔTₘ(MGB) | Tₘ(DNA) | ΔTₘ(DNA) | Discrimination |
| TCAGTGATTATTAT/MGB/ | No | 71.6 | 0.0 | 48.6 | 0.0 | 0.0 |
| | Mismatch | | | | | |
| TCAGTGATTATTAT/MGB/ | T/G, 1 | 71.5 | −0.1 | 47.6 | −1.0 | −0.9 |
| TCAGTGATTATTAT/MGB/ | A/G, 2 | 70.8 | −0.8 | 46.1 | −2.5 | −1.7 |
| TCAGTGATTATTAT/MGB/ | T/G, 3 | 65.7 | −5.9 | 43.4 | −5.2 | 0.7 |
| TCAGTGATTATTAT/MGB/ | T/G, 4 | 61.5 | −10.1 | 39.7 | −8.9 | 1.2 |
| TCAGTGATTATTAT/MGB/ | A/G, 5 | 59.1 | −12.5 | 41.7 | −6.9 | 5.6 |
| TCAGTGATTATTAT/MGB/ | T/G, 6 | 58.5 | −13.1 | 41.9 | −6.7 | 6.4 |
| TCAGTGATTATTAT/MGB/ | T/G, 7 | 60.1 | −11.5 | 39.2 | −9.4 | 2.1 |
| TCAGTGATTATTAT/MGB/ | A/G, 8 | 64.4 | −7.2 | 43.1 | −5.5 | 1.7 |
| TCAGTGATTATTAT/MGB/ | G/G, 9 | 59.7 | −11.9 | 34.9 | −13.7 | −1.8 |
| TCAGTGATTATTAT/MGB/ | T/G, 10 | 66.8 | −4.8 | 43.3 | −5.3 | −0.5 |
| TCAGTGATTATTAT/MGB/ | G/G, 11 | 64.1 | −7.5 | 40.1 | −8.5 | −1.0 |
| TCAGTGATTATTAT/MGB/ | A/A, 12 | 68.4 | −3.2 | 41.0 | −7.6 | −4.4 |
| TCAGTGATTATTAT/MGB/ | C/C, 13 | 69.1 | −2.5 | 43.3 | −5.3 | −2.8 |
| TCAGTGATTATTAT/MGB/ | T/C, 14 | 71.5 | −0.1 | 47.7 | −0.9 | −0.8 |

Table 12 presents discrimination data generated by varying the size and position of a bulge in a target sequence. The probe sequence used was 5′-TCAGTGATTATTAT/MGB/(SEQ ID: 155) and the target sequence was 3′-AGTCACTAXATAYATA-5′, where “X” and “Y” represent the position of the bulge identities provided in the “Bulge base(s)” column of Table 12. Position “X” is identified as position 7, as the bulged base(s) at position “X” are flanked by nucleotides complementary to the nucleotides at positions 6 and 7 of the probe (as measured from the MGB moiety/3′ end of the probe sequence). Similarly, position “Y” is identified as position 4, as the bulged base(s) at position “Y” are flanked by nucleotides complementary to the nucleotides at positions 3 and 4 of the probe (as measured from the MGB moiety/3′ end of the probe sequence). The impact of each bulge identity on discrimination was measured for both bulge positions. Sequence information for target sequences including a “CCC” bulge is given by SEQ IDs: 170-171; sequence information for target sequences including a “CC” bulge is given by SEQ IDs: 172-173; sequence information for target sequences including a “C” bulge is given by SEQ IDs: 174-175; sequence information for target sequences including a “T” bulge is given by SEQ IDs: 176-177; and sequence information for target sequences including a “A” bulge is given by SEQ IDs: 178-179.

As shown in Table 12, the addition of an MGB moiety increased discrimination where the target strand bulge was located at position “X.” Conversely, the addition of an MGB moiety did not significantly impact discrimination where the target strand bulge was located at position “Y.” In particular, for bulges at position “Y,” the addition of MGB yielded a positive increase in discrimination for only large bulges (i.e., for bulges “CCC” and “CC”) and yielded a small decrease in discrimination for small bulges (i.e., for bulges “C,” “T,” and “A”). Further, the magnitude of discrimination of the MGB-containing probe (i.e., ΔTₘ(MGB)) was at least 8 °C larger when the bulge is located in position “X” than when the bulge was located in position “Y.”

Notably, the data in Table 12 also indicates that bulge identity does not significantly impact discrimination. All three single nucleotide bulges (i.e., bulges “C,” “T,” and “A”) yielded similar discrimination at both position “X” and position “Y.”

TABLE 12
Effect of melting temperatures and discrimination (°C) of bulge size, location,
and identity of nucleotide for probe 5′ TCAGTGATTATTAT/MGB/and target
3′ AGTCACTAXATAYATA 5′. The target DNA strand contained a bulge either
at position 7 (X) or at position 4 (Y), where position 1 is the target sequence
nucleotide complementary to the probe position adjacent to the MGB moiety.

| | Bulge at position 7 (base X) | | | Bulge at position 4 (base Y) | | |
|---|---|---|---|---|---|---|
| | | | Increase of | | | Increase of |
| Bulge | ΔTₘ(MGB) | ΔTₘ(DNA) | Discrimination | ΔTₘ(MGB) | ΔTₘ(DNA) | Discrimination |
| base(s) | (°C) | (°C) | (°C) | (°C) | (°C) | (°C) |
| CCC | −29.3 | −18.3 | 11.0 | −9.3 | −8.4 | 0.9 |
| CC | −23.2 | −15.0 | 8.2 | −8.2 | −7.8 | 0.4 |
| C | −13.4 | −8.8 | 4.6 | −4.8 | −5.1 | −0.3 |
| T | −13.6 | −9.1 | 4.5 | −4.0 | −4.9 | −0.9 |
| A | −11.8 | −8.5 | 3.3 | −0.9 | −3.1 | −2.2 |

Table 13 presents discrimination data generated by varying the buffer used to generate discrimination data. The difference in melting temperature between reference-matched duplexes and bulged or mismatched duplexes were measured for bulged duplexes having a bulge in the probe sequence, bulged duplexes having a bulge in the target sequence and mismatched duplexes, as is indicated by the headings within Table 13 (i.e., within the “Sequence”) column. The identity and position of the bulged base are provided in the “Bulge base” column. Discrimination was measured for MGB-containing probes and non-MGB-containing probes for two different buffers. The buffers were selected to have different cations (i.e., and therefore different counterions for DNA in solution). In particular, the data in Table 13 was generated for a buffer containing 1M Na⁺ and also for IDT PCR buffer, which contains Mg²⁺ as a primary cation. ΔTₘ(MGB) and ΔTₘ(DNA) were generated by comparing the melting temperature of the reference-matched duplex with the melting temperature of corresponding the bulged or mismatched duplex, described with respect to the data of Tables 9-12. The probe used to collect data for duplexes including a bulge in the probe is given by SEQ ID: 154; the target sequences used to collect data for duplexes including a bulge in the target are given by SEQ ID: 175 and SEQ ID: 174; and the probe used to collect mismatch duplex data is given by SEQ ID: 155.

The data in Table 13 indicates that buffer (and, in particular, buffer cation selection) does not have a significant impact on discrimination for bulged duplexes where the bulge is in the probe sequence or the target sequence. The data in Table 13 also indicates that buffer does not have a significant impact on discrimination for mismatched duplexes. In particular, the duplex stability trends are the same and similar bulge discriminations are observed in both buffers. The differences in discrimination are minor and are generally within the error of experimentally measured ΔTₘ values (±0.6 °C).

TABLE 13
Effect of different buffer solutions on melting temperatures and discrimination
(°C) of MGB-containing oligonucleotide probes hybridized to a target DNA sequence.
The underscores (“_”) in the oligonucleotide probe sequences indicate the position
of the bulge in the probe or target sequence (according to the headings in the
table) when the probe and target sequence are hybridized.

| | | ΔTₘ(MGB) | | ΔTₘ(DNA) | |
|---|---|---|---|---|---|
| | Bulge or | (°C) | | (°C) | |
| | Mismatch | 1M Na⁺ | IDT PCR | 1M Na⁺ | IDT PCR |
| Sequence | base | buffer | buffer | buffer | buffer |
| Bulge in the probe | | | | | |
| 5′-GTTCAGCTCAGTGT/MGB/ | C6 | −16.9 | −16.3 | −15.0 | −14.3 |
| 5′-GTTCAGCTCAGTGT/MGB/ | T7 | −8.9 | −8.5 | −12.4 | −11.0 |
| Bulge in the target | | | | | |
| 3′-AGTCACTAATACATA | C4 | −4.8 | −4.8 | −5.1 | −5.8 |
| 3′-AGTCACTACATAATA | C7 | −13.4 | −12.1 | −8.8 | −8.0 |
| Mismatch | | | | | |
| 5′-TCAGTGATTATTAT/MGB/ | A/G, 2 | −0.8 | −2.0 | −2.5 | −2.8 |
| 5′-TCAGTGATTATTAT/MGB/ | T/G, 6 | −13.1 | −10.5 | −6.7 | −5.2 |

### Design of MGB-Containing Oligonucleotide Probes

The data provided in Tables 9-13 and described herein can be used to improve the design and synthesis of MGB-containing probes to improve and/or maximize discrimination.

For oligonucleotides used to detect mutant alleles including insertions, the data from Table 9 suggests constructing the oligonucleotide probe such that the bulge begins five or six nucleotides from a 3′ MGB moiety (i.e., spaced four or five nucleotides, respectively, from the 3′ end of the probe) to increase and/or maximize discrimination. While the aforementioned example refers to oligonucleotide probes for detecting mutants having an insertion relative to the wildtype, the above-articulated oligonucleotide design principle can be used to design oligonucleotide probes for other suitable examples where the probe includes a larger number of residues but is at least partially complementary to another, non-reference matched sequence. For example, the above designs (i.e., placing the bulge to begin 5 or 6 nucleotides from the 3′ end) can also be used to improve discrimination for oligonucleotides used to detect deletion-insertion mutants where the insertion is greater in length than the deletion. The above designs are yet further applicable to oligonucleotides used to detect a segment of a wildtype genomic sequence in a mixed sample including both the wildtype and a mutant allele including a deletion (i.e., such that the probe would preferentially bind to the wildtype sequence and would form a bulge when duplexed to the mutant allele).

For oligonucleotides used to detect a mutant allele including a deletion relative to a wildtype allele, the data of Table 10 suggests positioning adjacent nucleotides complementary to the closing base pairs (i.e., the base pairs flanking the bulged residues in the complementary sequence) four to nine nucleotides, inclusive, from the 3′ end of the probe to improve and/or maximize discrimination. In at least some examples, oligonucleotide probes will have the highest discrimination when the bases complementary to the closing base pairs are five to eight nucleotides, inclusive, from the 3′ end of the probe. Based on the data presented in Table 12, the aforementioned oligonucleotide probe designs are not dependent on the size of the bulge in the complementary strand and are applicable to probes where the bulge in the complementary strand (e.g., the deletion) is one, two, or three nucleotides.

The aforementioned examples generally refer to oligonucleotide probes for detecting mutant alleles having deletion relative to the wildtype, but can be used to design oligonucleotide probes for other suitable examples where the probe includes a smaller number of residues than but is at least partially complementary to another, non-reference matched sequence. For example, the same probe design principles can be used to improve discrimination for oligonucleotides used to detect deletion-insertion mutants where the insertion includes fewer nucleotides in length than the deletion. The above designs are yet further applicable to oligonucleotides used to detect a segment of a wildtype genomic sequence in a mixed sample including both the wildtype and a mutant allele including an insertion (i.e., such that the probe would preferentially bind to the wildtype sequence and the mutant allele would form a bulge when duplexed to the probe).

According to the data presented in Table 11, the discrimination of oligonucleotide probes for detection of mutant alleles including substitutions relative to corresponding wildtype alleles can be improved and/or maximized by positioning the 3′ end of the mismatch region (e.g., the internal loop) five or six nucleotides from the 3′ end of the probe (i.e., from the MGB moiety), such that the mismatch region is spaced from the 3′ end of the probe by four of five nucleotides, respectively.

Notably, according to the data presented in Table 13, the discrimination of MGB-containing oligonucleotide probes is not significantly impacted by buffer composition. As such, the aforementioned designs (i.e., for detecting deletions, insertions, substitutions, various deletion-insertions, etc.) can be used for oligonucleotide probe experiments in any suitable buffer.

More generally, the design principles articulated based on the collective data of Tables 9-13 can be condensed to a set of design principles for oligonucleotide probes that are generally depicted in FIG. 8. FIG. 8 is a schematic depiction of probe 700, which is an MGB-containing oligonucleotide probe. Further, as FIG. 8 is a schematic representation meant to facilitate discussion of design principles for oligonucleotide probes, all nucleotide identities are variable and accordingly are indicated by the letter “N.” In FIG. 8, ellipses (“ . . . ”) indicate that additional nucleotides may be present, as discussed in more detail subsequently.

Probe 700 includes fully complementary regions 710 and 720, which have nucleotide sequences that are selected to be fully complementary to both a mutant allele and the corresponding wildtype allele. Fully complementary region 710 can be any suitable length and includes at least one nucleotide and fully complementary region 720 can be 3 to 8 bases long, as discussed in more detail subsequently. According to the data presented in FIG. 4, discussed previously, in some examples it can advantageously improve discrimination for fully complementary region to include 3 nucleotides. Fully complementary regions 710 and 720 adjacently flank semi-complementary region 730 (i.e., are adjacent to the 5′ and 3′ ends, respectively, of semi-complementary region 730), which includes at least one nucleotide but can have a variable length as described subsequently. An MGB moiety is attached to probe 700 at the 3′ end of fully complementary region 720.

Semi-complementary region 730 includes one or more nucleotides that are fully complementary to the mutant allele and that are not fully complementary to the wildtype allele. Where the mutant allele includes an insertion, the bases of semi-complementary region 730 are selected to be complementary to the inserted bases and form a bulge when the probe is duplexed to the wildtype allele. Where the mutant allele includes one or more consecutive substitutions, the bases of semi-complementary region 730 are selected to match the sequence of the mutant allele, such that semi-complementary region 730 forms a mismatch region when the probe is duplexed to the wildtype allele. Where the mutant allele includes a deletion, the nucleotides of semi-complementary region 730 are selected to include two bases that are complementary to the bases flanking the 5′ and 3′ ends of the deleted nucleotides, such that semi-complementary region 730 is complementary to the closing base pairs of a bulge formed in a duplex with the wildtype sequence.

The length of fully complementary region 720 is chosen to position semi-complementary region 730—and thus the 3′ end of any bulge, internal loop, or base pairs to closing bases of a loop in a complementary sequence—four to nine bases from the MGB moiety attached to the 3′ end of fully complementary region 720. As such, fully complementary region 720 is three, four, five, six, seven, or eight nucleotides long. To further improve discrimination, where the mutant allele includes an insertion or a substitution, fully complementary region 720 can be four or five bases long (according to the data presented in Tables 9 and 11, respectively). Where the mutant allele includes a deletion, fully complementary region 720 can be four, five, six, or seven bases long to improve discrimination. To yet further improve discrimination, where the mutant allele includes a deletion, fully complementary region 720 can be six or seven bases long (according to the data presented in, e.g., Table 10).

The MGB moiety bound to the 3′ end of probes having the structure shown in FIG. 8 can be any suitable MGB moiety. In some examples, the probe can include a fluorophore and a quencher moiety such that the oligonucleotide probe can be used to perform a hydrolysis probe technique for detecting a target allele (e.g., 5′ nuclease assay), as described subsequently with respect to FIG. 9 and FIG. 10. In at least some of these examples, the fluorophore is attached to the 5′ end of the probe and the quencher is attached to the 3′ end of the probe.

In at least some examples, the MGB moiety can be MGB moiety 690, which is depicted in FIG. 7. MGB moiety 690 includes both MGB group 692 and quencher group 694 and is attached to the 3′ end via phosphate group 696. Quencher group 694 in FIG. 7 is the Eclipse® quencher (Glen Research) but is merely one example of a quencher group that can be included in an MGB moiety. In other examples, other suitable quenchers can be used, such as Black Hole Quencher-1 or an Iowa Black® quencher (Integrated DNA Technologies), among other options. Further, MGB group 692 is merely one example of an MGB group and, in other examples, other MGB groups capable of non-covalently interacting with the minor groove of oligonucleotide duplexes can be used in an MGB-containing oligonucleotide probe according to the present disclosure.

As described previously, MGB moiety 690 is 5-(6-(6-(6-(4-N-(3-Dimethoxytrityloxypropyl)-N-(4′-nitro-2′-chloroazobenzen-4-yl)-aminobutanoyl)-3,6,7,8-tetrahydropyrrolo[3,2-e]indole-2-carbonyl)-3,6,7,8-tetrahydropyrrolo[3,2-e]indole-2-carbonyl)-3,6,7,8-tetrahydropyrrolo[3,2-e]indole-2-carboxamido) pentanol and is one example of an MGB moiety compatible with the MGB design shown in FIG. 8. In other examples, however, any suitable MGB moiety can be used with the oligonucleotide design outlined in FIG. 8. For example, one or more of distamycin, netropsin, Hoechst 33258, and/or a pyrrole-imidazole polyamide can be used as the MGB moiety (Dervan P. B., and Edelson B. J., Recognition of the DNA minor groove by pyrrole-imidazole polyamides, Current Opinion in Structural Biology 13 (2003) 284-299; Fuchs J. E., Spitzer G. M., Javed A., Biela A., Kreutz C., Wellenzohn B., and Liedl K. A., Minor groove binders and drugs targeting proteins cover complementary regions in chemical shape space, J. Chem. Info. Model. 51 (2011) 2223-2232).

Where the mutant allele includes a deletion-insertion, the identity of bases of the semi-complementary region can be determined according to whether the deletion-insertion results in fewer nucleotides than the wildtype or the deletion-insertion results in a net gain of nucleotides as compared to the wildtype. Where there is a net loss of nucleotides, the oligonucleotide probes can be designed substantially similarly to the probes described above for detecting a deletion. Where there is a net gain of nucleotides, the oligonucleotide probes can be designed substantially similarly to the probes described above for detecting an insertion.

The probe design depicted in and discussed with respect to FIG. 8 is generally described herein as useful for detecting mutant alleles (as compared to wildtype alleles) for explanatory convenience, but, more generally, probe designs based on FIG. 8 can be adapted to detect any specific (i.e., target) allele of a biallelic or multiallelic genomic region, especially where the alleles differ by one or more insertion, deletion, deletion-insertion, and/or substitution mutations based on the structures formed between the oligonucleotide probe and the non-target allele. As a specific example, the probe design of FIG. 8 can be adapted to detect wildtype alleles in lieu of mutant alleles.

The aforementioned MGB-containing probe designs can be used to detect mutations in any suitable genetic material. For example, the oligonucleotide probe designs described herein can be used for oligonucleotide probes for detecting mutations in coding regions of genes, noncoding regions of genes, extragenic regions of a genome, any other suitable region of a genome, and/or any suitable mixture of the foregoing (e.g., a mutation that overlaps coding and noncoding regions, etc.).

The improved discrimination of probes generated according to the design outlined in FIG. 8 enables improved sensitivity for detecting a specific allele of a biallelic or multiallelic genomic region. In particular, the difference in melting temperature between the target allele and the non-target allele(s) is improved by the designs of the present disclosure. Moreover, the designs outlined herein are flexible and can be adapted to a detect wide variety of mutations and/or allelic variations, including substitutions, deletions, insertions, deletion-insertions, etc. The melting temperature of duplexes of MGB-containing probes can be determined via a known technique for use in designing experiments for detecting a specific, target allele.

Specific Allele Detection with Modified Probes

FIG. 9 is a flow diagram of method 800, which is an example of detecting a genomic alteration or variation according to the present disclosure. More specifically, method 800 enables the detection of a specific allele using an LNA-containing probe or MGB-containing probe according to the present disclosure. Method 800 includes steps 802-810 of providing a nucleotide sample (step 802), providing an oligonucleotide probe (step 804), heating the sample mixture (step 806), allowing the probe to hybridize to the target allele (step 808), and detecting the target allele based on the probe hybridization (step 810).

In step 802, a nucleotide sample is provided. The sample provided in step 802 can be any suitable nucleotide same and/or nucleotide-containing sample, and includes a genomic region of interest. The genomic region of interest is a biallelic region or multiallelic region, and otherwise can be any suitable genomic region, including a coding regions of a gene, a noncoding region of a gene, and extragenic region, any other suitable genomic region, and/or any suitable mixture of the foregoing (e.g., a mutation that overlaps coding and noncoding regions, etc.). The nucleotide sample can also be any suitable nucleotide sample, and can include any suitable nucleotide, including DNA or RNA, among other options, or any suitable mixture thereof.

In step 804, an oligonucleotide probe is provided. The oligonucleotide probe is a probe that is completely complementary to only one allele of the genomic region of interest (i.e., a target allele), and is a modified oligonucleotide probe according to the present disclosure. More specifically, the oligonucleotide probe is an LNA-containing oligonucleotide probe designed according to the design principles based on the data of Tables 1-7 and FIG. 4, or is an MGB-containing oligonucleotide probe designed according to the design principles based on the data of Tables 9-13. The oligonucleotide probe is added to the nucleotide sample to provide a nucleotide-containing sample mixture.

In step 806, the sample mixture is heated to denature the nucleotides provided in step 802. The sample mixture can be heated to any suitable denaturing temperature using any suitable apparatus. In at least some examples, the sample mixture is heated to a temperature in a range of 94 °C to 98 °C.

In step 808, the sample is brought to conditions that allow the probe added in step 804 to hybridize to the target allele (i.e., the allele of the biallelic or multiallelic region of interest to which the probe is completely complementary). In step 808, the sample mixture can be, for example, cooled to an annealing temperature below the melting temperature of the probe and the target allele and above the melting temperature(s) of the probe and the non-target allele(s). In step 808, the probe is able to hybridize (i.e., base pair) to the target allele if the target allele is present in the nucleotide sample provided in step 802. If the target allele is not present, then the probe is not able to hybridize to the non-target allele(s).

In step 810, the target allele is detected based on whether the probe was able to hybridize to the target allele in step 808. The detection method can be, for example, an in situ hybridization technique. In some of these examples, the probe can include covalently-attached fluorophore and the in situ hybridization technique can be a fluorescence in situ hybridization technique. In yet further examples, the target allele can be detected using a quantitative polymerase chain reaction (PCR) technique, such as a hydrolysis probe based quantitative PCR (qPCR) technique (i.e., a real-time PCR technique). The oligonucleotide probe included in step 810 can further include a fluorophore and a quencher moiety covalently attached to the oligonucleotide probe (e.g., a 5′ fluorophore and a 3′ quencher), and the hydrolysis probe technique can use, for example, a DNA polymerase with 5′-3′ exonuclease activity. In these examples, PCR amplification with a strand-displacing polymerase having 5′ exonuclease activity can degrade the oligonucleotide probe when complexed to the target allele, thereby freeing the fluorophore such that the fluorophore is no longer in proximity to the quencher and allowing fluorescence signal from the fluorophore to be detected using, e.g., a qPCR apparatus or another suitable apparatus. The aforementioned techniques of detecting probe hybridization are merely exemplary and, in other examples, other techniques for detecting probe hybridization can be used.

FIG. 10 is a flow diagram of method 900, which is another example of detecting a genomic alteration or variation according to the present disclosure. More specifically, method 900 enables the detection of a specific allele using an LNA-containing probe or MGB-containing probe according to the present disclosure. Method 900 is substantially similar to method 800 but includes separate steps for providing polymerase and target-specific primers as well as for amplifying the target genomic region. As such, method 900 can be understood as a variation of method 800 that is specific to hydrolysis probe detection techniques (i.e., for detection of probe hybridization, as discussed previously with respect to step 810 of method 800; FIG. 9). Method 900 includes steps 902-910 of providing a nucleotide sample (step 902), providing an oligonucleotide probe (step 904), providing polymerase and target-specific primers (step 905), heating the sample mixture (step 906), lowering the temperature of the sample mixture to an annealing temperature for the probe and the target-specific primers (step 908), amplifying the target genomic region with the target-specific primers (step 909), and detecting released fluorophore (step 910).

Step 902 and step 904 are substantially similar to step 802 and step 804, respectively, of method 800, and the description of step 802 and step 804 are applicable to step 902 and step 904, respectively. The oligonucleotide probes in step 904 each include a covalently-bound fluorophore and a covalently-bound quencher. The quencher is selected such that the fluorophore does not fluoresce when in proximity to the fluorophore (e.g., when both the quencher and fluorophore are covalently attached to an oligonucleotide probe) via, e.g., fluorescence resonance energy transfer (FRET) or another suitable quenching mechanism, such as static quenching.

In step 905, a polymerase and target-specific primers are added to the reaction mixture (i.e., the mixture of the oligonucleotide probe provided in step 904 and the nucleotide sample provided in step 902). The target-specific primers are selected to allow PCR amplification of the genomic region of interest (i.e., the biallelic or multiallelic region). The polymerase is able to at least partially degrade the oligonucleotide probe during amplification of the target allele to release the fluorophore and/or the quencher, disrupting the quenching mechanism and allowing fluorescence from the fluorophore to be detected in subsequent step 910. The polymerase can be a strand-displacing polymerase and can have, for example, 5′ exonuclease activity capable of degrading the oligonucleotide probe during PCR amplification of the target allele.

Step 906 is substantially similar to step 806 of method 800, and the description of step 806 herein is applicable to step 906.

In step 908, the temperature of the sample mixture is lowered to an annealing temperature for the probe and the target-specific primers. The temperature is selected in step 908 to allow the target-specific primers to hybridize to the target genomic region and to allow the probe to hybridize to the target allele. The annealing temperature is selected to be above the melting temperature for the probe and the non-target allele(s), such that the probe does not hybridize to the not target allele(s).

In step 909, the target genomic region is amplified by the polymerase provided in step 905 and using the target-specific primers also provided in step 905. The reaction mixture can optionally be brought to an extension temperature different from the annealing temperature used in step 908, and the extension temperature can be selected to improve processivity, activity, etc. of the polymerase during amplification. If the target allele is present in the nucleotide sample provided in step 902, the oligonucleotide probe is degraded by the polymerase (e.g., by the 5′ exonuclease activity of the polymerase) In step 909, the oligonucleotide probe is degraded to release fluorophore from physical proximity to the quencher. In at least some examples, the entire oligonucleotide probe is degraded by the polymerase (e.g., by the 5′ exonuclease activity of a suitable polymerase).

In step 910, fluorophore is detected. Detection of fluorophore indicates that the oligonucleotide probe was bound to the target allele and was at least partially degraded by the polymerase. As such, detection of fluorophore in step 910 indicates the presence of the target allele.

As indicated by the arrows in FIG. 10, steps 906-909 can be repeated multiple times (i.e., to thermocycle the PCR reaction) prior to detection in step 910. Additionally and/or alternatively, steps 906-910 can be repeated multiple times. Detection in step 910 can be performed after each iteration of step 909 and/or during any or all of steps 906-909 (i.e., via continuous or substantially continuous fluorescence signal monitoring) using, for example, a qPCR apparatus.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

### REFERENCES

1. Owczarzy R., You Y., Moreira B. G., Manthey J. A., Huang L., Behlke M. A., and Walder J. A., Effects of sodium ions on DNA duplex oligomers: Improved predictions of melting temperatures, Biochemistry 43 (2004) 3537-3554.
2. Fasman, G. D. (Ed.) (1975) Handbook of Biochemistry and Molecular Biology, Vol. I, p 589, CRC Press, Boca Raton, FL.
3. Cantor, C. R., Warshaw, M. M., and Shapiro, H. (1970) Oligonucleotide interactions. III. Circular dichroism studies of the conformation of deoxyoligonucleotides, Biopolymers 9, 1059-1077.
4. Li Y., and Agrawal S., Oligonucleotides containing G.A pairs: Effect of flanking sequences on structure and stability, Biochemistry 34 (1995) 10056-10062.
5. Owczarzy R., You Y., Growth C. L., and Tataurov A. V., Stability and mismatch discrimination of locked nucleic acid-DNA duplexes, Biochemistry 50 (2011) 9352-9367.
6. Dervan P. B., and Edelson B. J., Recognition of the DNA minor groove by pyrrole-imidazole polyamides, Current Opinion in Structural Biology 13 (2003) 284-299.
7. Fuchs J. E., Spitzer G. M., Javed A., Biela A., Kreutz C., Wellenzohn B., and Liedl K. A., Minor groove binders and drugs targeting proteins cover complementary regions in chemical shape space, J. Chem. Info. Model. 51 (2011) 2223-2232.

The following are non-exclusive descriptions of possible embodiments of the present invention.

### DISCUSSION OF POSSIBLE EMBODIMENTS

An embodiment of an oligonucleotide probe for detecting a genomic alteration including a first complementary region, a second complementary region, and a semi-complementary region. The first complementary region includes at least three nucleotides that are completely complementary to a mutant allele of a portion of a genomic sequence, the includes at least three nucleotides that are completely complementary to the mutant allele and to the wild type allele, and the semi-complementary region is adjacently flanked by the first complementary region and the second complementary region. The nucleotides of the semi-complementary region are completely complementary to the mutant allele and are not completely complementary to the wild type allele, the first complementary region comprises a first locked nucleic acid adjacent to the semi-complementary region, the second complementary region comprises a second locked nucleic acid adjacent to the semi-complementary region, and the semi-complementary region comprises a third locked nucleic acid.

The oligonucleotide probe of the preceding paragraph can optionally include, additionally and/or alternatively, any one or more of the following features, configurations and/or additional components:

An oligonucleotide probe for detecting a genomic alteration including a first complementary region, a second complementary region, and a semi-complementary region. The first complementary region includes at least three nucleotides that are completely complementary to a mutant allele of a portion of a genomic sequence, the includes at least three nucleotides that are completely complementary to the mutant allele and to the wild type allele, and the semi-complementary region is adjacently flanked by the first complementary region and the second complementary region. The nucleotides of the semi-complementary region are completely complementary to the mutant allele and are not completely complementary to the wild type allele, the first complementary region comprises a first locked nucleic acid adjacent to the semi-complementary region, the second complementary region comprises a second locked nucleic acid adjacent to the semi-complementary region, and the semi-complementary region comprises a third locked nucleic acid.

A further embodiment of the foregoing oligonucleotide probe, wherein the semi-complementary region consists of two nucleotides or one nucleotide.

A further embodiment of the foregoing oligonucleotide probe, wherein the semi-complementary region consists of the third locked nucleic acid and a fourth locked nucleic acid.

A further embodiment of the foregoing oligonucleotide probe, wherein the mutant allele contains one of a substitution, an insertion, and a deletion.

A further embodiment of the foregoing oligonucleotide probe, wherein the nucleotides of the semi-complementary region is completely complementary to a mutation-containing region of the mutant allele containing the one of the substitution, the insertion, and the deletion, the at least three nucleotides of the first complementary region are completely complementary to a first region adjacent to the mutation-containing region, and the at least three nucleotides of the second complementary region are completely complementary to a second region adjacent to the mutation-containing region.

A further embodiment of the foregoing oligonucleotide probe, wherein the mutant allele contains a single-nucleotide substitution, the semi-complementary region consists of one locked nucleic acid complementary to the single-nucleotide substitution, the at least three nucleotides of the first complementary region are completely complementary to a first region of the mutant allele adjacent to the single-nucleotide substitution, and the at least three nucleotides of the second complementary region are completely complementary to a second region of the mutant allele adjacent to the single-nucleotide substitution.

A further embodiment of the foregoing oligonucleotide probe, wherein the mutant allele contains consecutive first and second nucleotide substitutions, the semi-complementary region consists of two locked nucleic acids that are complementary the consecutive first and second nucleotide substitutions, the at least three nucleotides of the first complementary region are completely complementary to a first region of the mutant allele adjacent to the first nucleotide substitution, and the at least three nucleotides of the second complementary region are completely complementary to a second region of the mutant allele adjacent to the second nucleotide substitution.

A further embodiment of the foregoing oligonucleotide probe, wherein the mutant allele contains an insertion of at least one nucleotide, the semi-complementary region consists of one or more locked nucleic acids that are completely complementary to the at least one nucleotide, the at least three nucleotides of the first complementary region are completely complementary to a first region of the mutant allele adjacent to the at least one nucleotide, and the at least three nucleotides of the complementary region are completely complementary to a second region of the mutant allele adjacent to the at least one nucleotide.

A further embodiment of the foregoing oligonucleotide probe, wherein the mutant allele contains a deletion of at least one deleted nucleotide, the semi-complementary region is complementary to: a first nucleotide adjacently flanking a location the at least one deleted nucleotide, and a second nucleotide adjacently flanking the location of at least one deleted nucleotide, the first complementary region is completely complementary to a first region adjacently flanking the first nucleotide, and the second complementary region is completely complementary to a second region adjacently flanking the second nucleotide.

A further embodiment of the foregoing oligonucleotide probe, wherein the mutant allele contains a deletion-insertion including at least two mutant nucleotides, the semi-complementary region consists of two or more locked nucleic acids that are completely complementary to the at least two mutant nucleotides, the first complementary region is completely complementary to a first region adjacently flanking the at least two mutant nucleotides, and the second complementary region is completely complementary to a second region adjacently flanking the at least two mutant nucleotides.

A further embodiment of the foregoing oligonucleotide probe, and further comprising a fluorophore attached to a first end of the oligonucleotide probe; and a quencher attached to a second end of the oligonucleotide probe.

A further embodiment of the foregoing oligonucleotide probe, wherein the mutant allele contains one of an insertion, a deletion, and a deletion-insertion; and a portion of the mutant allele containing the one of the insertion, the deletion, and the deletion-insertion differs by at least one nucleotide and up to ten nucleotides from a corresponding portion of the wild type allele.

An embodiment an oligonucleotide probe for detecting a genomic alteration including a first complementary region, a second complementary region, and a semi-complementary region. The first complementary region includes at least three nucleotides that are completely complementary to a mutant allele of a portion of a genomic sequence and to a wild type allele of the portion of the genomic sequence, the second complementary region includes at least three nucleotides that are completely complementary to the mutant allele and to the wild type allele, and the semi-complementary region includes three locked nucleic acid residues that are completely complementary to the mutant allele and that are not completely complementary to the wild type allele.

The oligonucleotide probe of the preceding paragraph can optionally include, additionally and/or alternatively, any one or more of the following features, configurations and/or additional components:

An oligonucleotide probe for detecting a genomic alteration including a first complementary region, a second complementary region, and a semi-complementary region. The first complementary region includes at least three nucleotides that are completely complementary to a mutant allele of a portion of a genomic sequence and to a wild type allele of the portion of the genomic sequence, the second complementary region includes at least three nucleotides that are completely complementary to the mutant allele and to the wild type allele, and the semi-complementary region includes three locked nucleic acid residues that are completely complementary to the mutant allele and that are not completely complementary to the wild type allele.

A further embodiment of the foregoing oligonucleotide probe, wherein the first complementary region does not contain a locked nucleic acid residue.

A further embodiment of the foregoing oligonucleotide probe, wherein both the first complementary region and the second complementary region do not contain locked nucleic acid residues.

A further embodiment of the foregoing oligonucleotide probe, wherein the semi-complementary region consists of three or four locked nucleic acid residues.

An embodiment of a method of detecting a mutant allele of a gene including providing a sample containing the mutant allele and a wildtype allele of the gene, providing the sample with an oligonucleotide probe, heating the sample to denature the mutant allele and the wildtype allele, lowering the temperature of the sample, after heating the sample, to an annealing temperature below a first melting temperature between the oligonucleotide probe and the mutant allele and above a second melting temperature between the oligonucleotide probe and the wild type allele, hybridizing the probe to the mutant allele at the annealing temperature to form a mutant allele and oligonucleotide probe hybrid, and detecting the mutant allele by detecting the mutant allele and oligonucleotide probe hybrid. The oligonucleotide probe includes a first complementary region, a second complementary region, and a semi-complementary region. The first complementary region includes at least three nucleotides that are completely complementary to a mutant allele of a portion of a genomic sequence, the includes at least three nucleotides that are completely complementary to the mutant allele and to the wild type allele, and the semi-complementary region is adjacently flanked by the first complementary region and the second complementary region. The nucleotides of the semi-complementary region are completely complementary to the mutant allele and are not completely complementary to the wild type allele, the first complementary region comprises a first locked nucleic acid adjacent to the semi-complementary region, the second complementary region comprises a second locked nucleic acid adjacent to the semi-complementary region, and the semi-complementary region comprises a third locked nucleic acid.

The method of the preceding paragraph can optionally include, additionally and/or alternatively, any one or more of the following features, configurations and/or additional components:
A method of detecting a mutant allele of a gene including providing a sample containing the mutant allele and a wildtype allele of the gene, providing the sample with an oligonucleotide probe, heating the sample to denature the mutant allele and the wildtype allele, lowering the temperature of the sample, after heating the sample, to an annealing temperature below a first melting temperature between the oligonucleotide probe and the mutant allele and above a second melting temperature between the oligonucleotide probe and the wild type allele, hybridizing the probe to the mutant allele at the annealing temperature to form a mutant allele and oligonucleotide probe hybrid, and detecting the mutant allele by detecting the mutant allele and oligonucleotide probe hybrid. The oligonucleotide probe includes a first complementary region, a second complementary region, and a semi-complementary region. The first complementary region includes at least three nucleotides that are completely complementary to a mutant allele of a portion of a genomic sequence, the includes at least three nucleotides that are completely complementary to the mutant allele and to the wild type allele, and the semi-complementary region is adjacently flanked by the first complementary region and the second complementary region. The nucleotides of the semi-complementary region are completely complementary to the mutant allele and are not completely complementary to the wild type allele, the first complementary region comprises a first locked nucleic acid adjacent to the semi-complementary region, the second complementary region comprises a second locked nucleic acid adjacent to the semi-complementary region, and the semi-complementary region comprises a third locked nucleic acid.

A further embodiment of the foregoing method, wherein the first complementary region of the oligonucleotide probe comprises a second locked nucleic acid adjacent to the semi-complementary region, and the second complementary region of the oligonucleotide probe comprises a third locked nucleic acid adjacent to the semi-complementary region.

A further embodiment of the foregoing method, and further comprising providing the sample with target-specific primers capable of hybridizing with both the wildtype allele and the mutant allele; and providing the sample with a polymerase; wherein detecting the mutant allele and oligonucleotide probe hybrid comprises producing mutant amplicons by amplifying at least a portion of the mutant allele complementary to the oligonucleotide probe with the target-specific primers and the polymerase.

A further embodiment of the foregoing method, wherein the oligonucleotide probe includes a fluorophore attached to a first end of the oligonucleotide probe and a quencher attached to a second end of the oligonucleotide probe, amplifying the at least the portion of the mutant allele complementary to the oligonucleotide probe breaks a chemical bond to produce freed fluorophore, and detecting the mutant allele and oligonucleotide probe hybrid comprises detecting the freed fluorophore.

A further embodiment of the foregoing method, wherein: the oligonucleotide probe includes a fluorophore attached to a first end of the oligonucleotide probe and a quencher attached to a second end of the oligonucleotide probe, amplifying the at least the portion of the mutant allele complementary to the oligonucleotide probe breaks a chemical bond to produce freed fluorophore, and detecting the mutant allele and oligonucleotide probe hybrid comprises detecting the freed fluorophore.

A further embodiment of the foregoing method, wherein the mutant allele contains one of a substitution, an insertion, a deletion, and a deletion-insertion.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. An oligonucleotide probe for detecting a genomic alteration, the oligonucleotide probe comprising:
a first complementary region including at least three nucleotides completely complementary to a mutant allele of a portion of a genomic sequence and to a wild type allele of the portion of the genomic sequence;
a second complementary region including at least three nucleotides completely complementary to the mutant allele and to the wild type allele; and
a semi-complementary region adjacently flanked by the first complementary region and the second complementary region,
wherein:
nucleotides of the semi-complementary region are completely complementary to the mutant allele and are not completely complementary to the wild type allele,
the first complementary region comprises a first locked nucleic acid adjacent to the semi-complementary region,
the second complementary region comprises a second locked nucleic acid adjacent to the semi-complementary region, and
the semi-complementary region comprises a third locked nucleic acid.

2. The oligonucleotide probe of claim 1, wherein the semi-complementary region consists of two nucleotides or one nucleotide.

3. The oligonucleotide probe of claim 1, wherein the semi-complementary region consists of the third locked nucleic acid and a fourth locked nucleic acid.

4. The oligonucleotide probe of claim 1, wherein the mutant allele contains one of a substitution, an insertion, and a deletion.

5. The oligonucleotide probe of claim 4, wherein:
the nucleotides of the semi-complementary region is completely complementary to a mutation-containing region of the mutant allele containing the one of the substitution, the insertion, and the deletion,
the at least three nucleotides of the first complementary region are completely complementary to a first region adjacent to the mutation-containing region, and
the at least three nucleotides of the second complementary region are completely complementary to a second region adjacent to the mutation-containing region.

6. The oligonucleotide probe of claim 1, wherein:
the mutant allele contains a single-nucleotide substitution,
the semi-complementary region consists of one locked nucleic acid complementary to the single-nucleotide substitution,
the at least three nucleotides of the first complementary region are completely complementary to a first region of the mutant allele adjacent to the single-nucleotide substitution, and
the at least three nucleotides of the second complementary region are completely complementary to a second region of the mutant allele adjacent to the single-nucleotide substitution.

7. The oligonucleotide probe of claim 1, wherein:
the mutant allele contains consecutive first and second nucleotide substitutions,
the semi-complementary region consists of two locked nucleic acids that are complementary the consecutive first and second nucleotide substitutions,
the at least three nucleotides of the first complementary region are completely complementary to a first region of the mutant allele adjacent to the first nucleotide substitution, and
the at least three nucleotides of the second complementary region are completely complementary to a second region of the mutant allele adjacent to the second nucleotide substitution.

8. The oligonucleotide probe of claim 1, wherein:
the mutant allele contains an insertion of at least one nucleotide,
the semi-complementary region consists of one or more locked nucleic acids that are completely complementary to the at least one nucleotide,
the at least three nucleotides of the first complementary region are completely complementary to a first region of the mutant allele adjacent to the at least one nucleotide, and
the at least three nucleotides of the complementary region are completely complementary to a second region of the mutant allele adjacent to the at least one nucleotide.

9. The oligonucleotide probe of claim 1, wherein:
the mutant allele contains a deletion of at least one deleted nucleotide,
the semi-complementary region is complementary to:
a first nucleotide adjacently flanking a location the at least one deleted nucleotide, and
a second nucleotide adjacently flanking the location of at least one deleted nucleotide,
the first complementary region is completely complementary to a first region adjacently flanking the first nucleotide, and
the second complementary region is completely complementary to a second region adjacently flanking the second nucleotide.

10. The oligonucleotide probe of claim 1, wherein:
the mutant allele contains a deletion-insertion including at least two mutant nucleotides,
the semi-complementary region consists of two or more locked nucleic acids that are completely complementary to the at least two mutant nucleotides,
the first complementary region is completely complementary to a first region adjacently flanking the at least two mutant nucleotides, and
the second complementary region is completely complementary to a second region adjacently flanking the at least two mutant nucleotides.

11. The oligonucleotide probe of claim 1, and further comprising:
a fluorophore attached to a first end of the oligonucleotide probe; and
a quencher attached to a second end of the oligonucleotide probe.

12. The oligonucleotide probe of claim 1, wherein:
the mutant allele contains one of an insertion, a deletion, and a deletion-insertion; and
a portion of the mutant allele containing the one of the insertion, the deletion, and the deletion-insertion differs by at least one nucleotide and up to ten nucleotides from a corresponding portion of the wild type allele.

13. An oligonucleotide probe for detecting a genomic alteration, the oligonucleotide probe comprising:
a first complementary region including at least three nucleotides completely complementary to a mutant allele of a portion of a genomic sequence and to a wild type allele of the portion of the genomic sequence;
a second complementary region including at least three nucleotides completely complementary to the mutant allele and to the wild type allele; and
a semi-complementary region adjacently flanked by the first complementary region and the second complementary region, wherein the semi-complementary region includes three locked nucleic acid residues that are completely complementary to the mutant allele and that are not completely complementary to the wild type allele.

14. The oligonucleotide probe of claim 13, wherein the first complementary region does not contain a locked nucleic acid residue.

15. The oligonucleotide of claim 13, wherein both the first complementary region and the second complementary region do not contain locked nucleic acid residues.

16. The oligonucleotide of claim 15, wherein the semi-complementary region consists of three or four locked nucleic acid residues.

17. A method of detecting a genomic alteration, the method comprising:
providing a sample containing a mutant allele of a portion of a genomic sequence and a wildtype allele of the portion of the genomic sequence;
providing the sample with an oligonucleotide probe comprising:
a first complementary region including at least three nucleotides completely complementary to the mutant allele and to the wild type allele;
a second complementary region including at least three nucleotides completely complementary to the mutant allele and to the wild type allele; and
a semi-complementary region adjacently flanked by the first complementary region and the second complementary region,
wherein:
nucleotides of the semi-complementary region are completely complementary to the mutant allele and are not completely complementary to the wild type allele, and
the semi-complementary region comprises a first locked nucleic acid;
heating the sample to denature the mutant allele and the wildtype allele;
lower the temperature of the sample, after heating the sample, to an annealing temperature below a first melting temperature between the oligonucleotide probe and the mutant allele and above a second melting temperature between the oligonucleotide probe and the wild type allele;
hybridizing the probe to the mutant allele at the annealing temperature to form a mutant allele and oligonucleotide probe hybrid; and
detecting the mutant allele by detecting the mutant allele and oligonucleotide probe hybrid.

18. The method of claim 17, wherein:
the first complementary region of the oligonucleotide probe comprises a second locked nucleic acid adjacent to the semi-complementary region, and
the second complementary region of the oligonucleotide probe comprises a third locked nucleic acid adjacent to the semi-complementary region.

19. The method of claim 17, and further comprising:
providing the sample with target-specific primers capable of hybridizing with both the wildtype allele and the mutant allele; and
providing the sample with a polymerase;
wherein detecting the mutant allele and oligonucleotide probe hybrid comprises producing mutant amplicons by amplifying at least a portion of the mutant allele complementary to the oligonucleotide probe with the target-specific primers and the polymerase.

20. The method of claim 19, wherein:
the oligonucleotide probe includes a fluorophore attached to a first end of the oligonucleotide probe and a quencher attached to a second end of the oligonucleotide probe,
amplifying the at least the portion of the mutant allele complementary to the oligonucleotide probe breaks a chemical bond to produce freed fluorophore, and
detecting the mutant allele and oligonucleotide probe hybrid comprises detecting the freed fluorophore.


SEQUENCE LISTING (190 sequences)

SEQ ID NO: 1 - DNA, 15 bp, synthetic construct
  source 1..15: mol_type = other DNA
aagtccacagtgatc

SEQ ID NO: 2 - DNA, 15 bp, synthetic construct
  source 1..15: mol_type = other DNA
  modified_base 8: mod_base = OTHER; note = Locked Nucleic Acid
aagtccacagtgatc

SEQ ID NO: 3 - DNA, 15 bp, synthetic construct
  source 1..15: mol_type = other DNA
  modified_base 7: mod_base = OTHER; note = Locked Nucleic Acid
  modified_base 8: mod_base = OTHER; note = Locked Nucleic Acid
  modified_base 9: mod_base = OTHER; note = Locked Nucleic Acid
aagtccacagtgatc

SEQ ID NO: 4 - DNA, 15 bp, synthetic construct
  source 1..15: mol_type = other DNA
  modified_base 7: mod_base = OTHER; note = Locked Nucleic Acid
  modified_base 9: mod_base = OTHER; note = Locked Nucleic Acid
aagtccacagtgatc

SEQ ID NO: 5 - DNA, 15 bp, synthetic construct
  source 1..15: mol_type = other DNA
  modified_base 6: mod_base = OTHER; note = Locked Nucleic Acid
  modified_base 10: mod_base = OTHER; note = Locked Nucleic Acid
aagtccacagtgatc

SEQ ID NO: 6 - DNA, 15 bp, synthetic construct
  source 1..15: mol_type = other DNA
  modified_base 4: mod_base = OTHER; note = Locked Nucleic Acid
  modified_base 12: mod_base = OTHER; note = Locked Nucleic Acid
aagtccacagtgatc

SEQ ID NO: 7 - DNA, 15 bp, synthetic construct
  source 1..15: mol_type = other DNA
  modified_base 6: mod_base = OTHER; note = Locked Nucleic Acid
  modified_base 8: mod_base = OTHER; note = Locked Nucleic Acid
  modified_base 10: mod_base = OTHER; note = Locked Nucleic Acid
aagtccacagtgatc

SEQ ID NO: 8 - DNA, 15 bp, synthetic construct
  source 1..15: mol_type = other DNA
  modified_base 6: mod_base = OTHER; note = Locked Nucleic Acid
  modified_base 10: mod_base = OTHER; note = Locked Nucleic Acid
  modified_base 12: mod_base = OTHER; note = Locked Nucleic Acid
aagtccacagtgatc

SEQ ID NO: 9 - DNA, 15 bp, synthetic construct
  source 1..15: mol_type = other DNA
  modified_base 4: mod_base = OTHER; note = Locked Nucleic Acid
  modified_base 6: mod_base = OTHER; note = Locked Nucleic Acid
  modified_base 10: mod_base = OTHER; note = Locked Nucleic Acid
  modified_base 12: mod_base = OTHER; note = Locked Nucleic Acid
aagtccacagtgatc

SEQ ID NO: 10 - DNA, 15 bp, synthetic construct
  source 1..15: mol_type = other DNA
aagtccaaagtgatc

SEQ ID NO: 11 - DNA, 15 bp, synthetic construct
  source 1..15: mol_type = other DNA
  modified_base 8: mod_base = OTHER; note = Locked Nucleic Acid
aagtccaaagtgatc

SEQ ID NO: 12 - DNA, 15 bp, synthetic construct
  source 1..15: mol_type = other DNA
  modified_base 7..9: mod_base = OTHER; note = Locked Nucleic Acids
aagtccaaagtgatc

SEQ ID NO: 13 - DNA, 15 bp, synthetic construct
  source 1..15: mol_type = other DNA
  modified_base 7: mod_base = OTHER; note = Locked Nucleic Acid
  modified_base 9: mod_base = OTHER; note = Locked Nucleic Acid
aagtccaaagtgatc

SEQ ID NO: 14 - DNA, 12 bp, synthetic construct
  source 1..12: mol_type = other DNA
gagagcgaccac

SEQ ID NO: 15 - DNA, 12 bp, synthetic construct
  source 1..12: mol_type = other DNA
  modified_base 6: mod_base = OTHER; note = Locked Nucleic Acid
gagagcgaccac

SEQ ID NO: 16 - DNA, 12 bp, synthetic construct
  source 1..12: mol_type = other DNA
  modified_base 5..7: mod_base = OTHER; note = Locked Nucleic Acid
gagagcgaccac

SEQ ID NO: 17 - DNA, 12 bp, synthetic construct
  source 1..12: mol_type = other DNA
  modified_base 5: mod_base = OTHER; note = Locked Nucleic Acid
  modified_base 7: mod_base = OTHER; note = Locked Nucleic Acid
gagagcgaccac

SEQ ID NO: 18 - DNA, 12 bp, synthetic construct
  source 1..12: mol_type = other DNA
gagagtgaccac

SEQ ID NO: 19 - DNA, 12 bp, synthetic construct
  source 1..12: mol_type = other DNA
  modified_base 6: mod_base = OTHER; note = Locked Nucleic Acid
gagagtgaccac

SEQ ID NO: 20 - DNA, 12 bp, synthetic construct
  source 1..12: mol_type = other DNA
  modified_base 5..7: mod_base = OTHER; note = Locked Nucleic Acid
gagagtgaccac

SEQ ID NO: 21 - DNA, 12 bp, synthetic construct
  source 1..12: mol_type = other DNA
  modified_base 5: mod_base = OTHER; note = Locked Nucleic Acid
  modified_base 7: mod_base = OTHER; note = Locked Nucleic Acid
gagagtgaccac

SEQ ID NO: 22 - DNA, 12 bp, synthetic construct
  source 1..12: mol_type = other DNA
gagagagaccac

SEQ ID NO: 23 - DNA, 12 bp, synthetic construct
  source 1..12: mol_type = other DNA
  modified_base 6: mod_base = OTHER; note = Locked Nucleic Acid
gagagagaccac

SEQ ID NO: 24 - DNA, 12 bp, synthetic construct
  source 1..12: mol_type = other DNA
  modified_base 5..7: mod_base = OTHER; note = Locked Nucleic Acids
gagagagaccac

SEQ ID NO: 25 - DNA, 12 bp, synthetic construct
  source 1..12: mol_type = other DNA
  modified_base 5: mod_base = OTHER; note = Locked Nucleic Acid
  modified_base 7: mod_base = OTHER; note = Locked Nucleic Acid
gagagagaccac

SEQ ID NO: 26 - DNA, 12 bp, synthetic construct
  source 1..12: mol_type = other DNA
gagagggaccac

SEQ ID NO: 27 - DNA, 12 bp, synthetic construct
  source 1..12: mol_type = other DNA
  modified_base 6: mod_base = OTHER; note = Locked Nucleic Acid
gagagggaccac

SEQ ID NO: 28 - DNA, 12 bp, synthetic construct
  source 1..12: mol_type = other DNA
  modified_base 5..7: mod_base = OTHER; note = Locked Nucleic Acids
gagagggaccac

SEQ ID NO: 29 - DNA, 12 bp, synthetic construct
  source 1..12: mol_type = other DNA
  modified_base 5: mod_base = OTHER; note = Locked Nucleic Acid
  modified_base 7: mod_base = OTHER; note = Locked Nucleic Acid
gagagggaccac

SEQ ID NO: 30 - DNA, 16 bp, synthetic construct
  source 1..16: mol_type = other DNA
aagtccaccagtgatc

SEQ ID NO: 31 - DNA, 16 bp, synthetic construct
  source 1..16: mol_type = other DNA
  modified_base 8..9: mod_base = OTHER; note = Locked Nucleic Acids
aagtccaccagtgatc

SEQ ID NO: 32 - DNA, 16 bp, synthetic construct
  source 1..16: mol_type = other DNA
  modified_base 7..10: mod_base = OTHER; note = Locked Nucleic Acids
aagtccaccagtgatc

SEQ ID NO: 33 - DNA, 16 bp, synthetic construct
  source 1..16: mol_type = other DNA
  modified_base 7: mod_base = OTHER; note = Locked Nucleic Acid
  modified_base 10: mod_base = OTHER; note = Locked Nucleic Acid
aagtccaccagtgatc

SEQ ID NO: 34 - DNA, 17 bp, synthetic construct
  source 1..17: mol_type = other DNA
aagtccacccagtgatc

SEQ ID NO: 35 - DNA, 17 bp, synthetic construct
  source 1..17: mol_type = other DNA
  modified_base 8..10: mod_base = OTHER; note = Locked Nucleic Acid
aagtccacccagtgatc

SEQ ID NO: 36 - DNA, 17 bp, synthetic construct
  source 1..17: mol_type = other DNA
  modified_base 7..8: mod_base = OTHER; note = Locked Nucleic Acids
  modified_base 10..11: mod_base = OTHER; note = Locked Nucleic Acids
aagtccacccagtgatc

SEQ ID NO: 37 - DNA, 18 bp, synthetic construct
  source 1..18: mol_type = other DNA
aagtccaccccagtgatc

SEQ ID NO: 38 - DNA, 18 bp, synthetic construct
  source 1..18: mol_type = other DNA
  modified_base 8..11: mod_base = OTHER; note = Locked Nucleic Acids
aagtccaccccagtgatc

SEQ ID NO: 39 - DNA, 18 bp, synthetic construct
  source 1..18: mol_type = other DNA
  modified_base 7..8: mod_base = OTHER; note = Locked Nucleic Acids
  modified_base 11..12: mod_base = OTHER; note = Locked Nucleic Acids
aagtccaccccagtgatc

SEQ ID NO: 40 - DNA, 18 bp, synthetic construct
  source 1..18: mol_type = other DNA
  modified_base 7: mod_base = OTHER; note = Locked Nucleic Acid
  modified_base 9..10: mod_base = OTHER; note = Locked Nucleic Acids
  modified_base 12: mod_base = OTHER; note = Locked Nucleic Acid
aagtccaccccagtgatc

SEQ ID NO: 41 - DNA, 18 bp, synthetic construct
  source 1..18: mol_type = other DNA
  modified_base 9..10: mod_base = OTHER; note = Locked Nucleic Acids
aagtccaccccagtgatc

SEQ ID NO: 42 - DNA, 11 bp, synthetic construct
  source 1..11: mol_type = other DNA
gtggtcctctc

SEQ ID NO: 43 - DNA, 11 bp, synthetic construct
  source 1..11: mol_type = other DNA
  modified_base 6..7: mod_base = OTHER; note = Locked Nucleic Acids
gtggtcctctc

SEQ ID NO: 44 - DNA, 11 bp, synthetic construct
  source 1..11: mol_type = other DNA
  modified_base 5..8: mod_base = OTHER; note = Locked Nucleic Acids
gtggtcctctc

SEQ ID NO: 45 - DNA, 11 bp, synthetic construct
  source 1..11: mol_type = other DNA
  modified_base 5: mod_base = OTHER; note = Locked Nucleic Acid
  modified_base 8: mod_base = OTHER; note = Locked Nucleic Acid
gtggtcctctc

SEQ ID NO: 46 - DNA, 11 bp, synthetic construct
  source 1..11: mol_type = other DNA
gtggtcctctc

SEQ ID NO: 47 - DNA, 11 bp, synthetic construct
  source 1..11: mol_type = other DNA
  modified_base 6..7: mod_base = OTHER; note = Locked Nucleic Acids
gtggtcctctc

SEQ ID NO: 48 - DNA, 11 bp, synthetic construct
  source 1..11: mol_type = other DNA
  modified_base 5..8: mod_base = OTHER; note = Locked Nucleic Acids
gtggtcctctc

SEQ ID NO: 49 - DNA, 11 bp, synthetic construct
  source 1..11: mol_type = other DNA
  modified_base 5: mod_base = OTHER; note = Locked Nucleic Acid
  modified_base 8: mod_base = OTHER; note = Locked Nucleic Acid
gtggtcctctc

SEQ ID NO: 50 - DNA, 11 bp, synthetic construct
  source 1..11: mol_type = other DNA
gtggtcctctc

SEQ ID NO: 51 - DNA, 11 bp, synthetic construct
  source 1..11: mol_type = other DNA
  modified_base 6..7: mod_base = OTHER; note = Locked Nucleic Acids
gtggtcctctc

SEQ ID NO: 52 - DNA, 11 bp, synthetic construct
  source 1..11: mol_type = other DNA
  modified_base 5..8: mod_base = OTHER; note = Locked Nucleic Acids
gtggtcctctc

SEQ ID NO: 53 - DNA, 11 bp, synthetic construct
  source 1..11: mol_type = other DNA
  modified_base 5: mod_base = OTHER; note = Locked Nucleic Acid
  modified_base 8: mod_base = OTHER; note = Locked Nucleic Acid
gtggtcctctc

SEQ ID NO: 54 - DNA, 11 bp, synthetic construct
  source 1..11: mol_type = other DNA
gtggtcctctc

SEQ ID NO: 55 - DNA, 11 bp, synthetic construct
  source 1..11: mol_type = other DNA
  modified_base 6..7: mod_base = OTHER; note = Locked Nucleic Acids
gtggtcctctc

SEQ ID NO: 56 - DNA, 11 bp, synthetic construct
  source 1..11: mol_type = other DNA
  modified_base 5..8: mod_base = OTHER; note = Locked Nucleic Acids
gtggtcctctc

SEQ ID NO: 57 - DNA, 11 bp, synthetic construct
  source 1..11: mol_type = other DNA
  modified_base 5: mod_base = OTHER; note = Locked Nucleic Acid
  modified_base 8: mod_base = OTHER; note = Locked Nucleic Acid
gtggtcctctc

SEQ ID NO: 58 - DNA, 14 bp, synthetic construct
  source 1..14: mol_type = other DNA
gatcacttggactt

SEQ ID NO: 59 - DNA, 14 bp, synthetic construct
  source 1..14: mol_type = other DNA
  modified_base 7..8: mod_base = OTHER; note = Locked Nucleic Acids
gatcacttggactt

SEQ ID NO: 60 - DNA, 14 bp, synthetic construct
  source 1..14: mol_type = other DNA
  modified_base 6..9: mod_base = OTHER; note = Locked Nucleic Acids
gatcacttggactt

SEQ ID NO: 61 - DNA, 14 bp, synthetic construct
  source 1..14: mol_type = other DNA
  modified_base 6: mod_base = OTHER; note = Locked Nucleic Acid
  modified_base 9: mod_base = OTHER; note = Locked Nucleic Acid
gatcacttggactt

SEQ ID NO: 62 - DNA, 14 bp, synthetic construct
  source 1..14: mol_type = other DNA
  modified_base 3..4: mod_base = OTHER; note = Locked Nucleic Acids
gatcacttggactt

SEQ ID NO: 63 - DNA, 14 bp, synthetic construct
  source 1..14: mol_type = other DNA
gatcacttggactt

SEQ ID NO: 64 - DNA, 14 bp, synthetic construct
  source 1..14: mol_type = other DNA
  modified_base 7..8: mod_base = OTHER; note = Locked Nucleic Acids
gatcacttggactt

SEQ ID NO: 65 - DNA, 14 bp, synthetic construct
  source 1..14: mol_type = other DNA
  modified_base 6..9: mod_base = OTHER; note = Locked Nucleic Acids
gatcacttggactt

SEQ ID NO: 66 - DNA, 14 bp, synthetic construct
  source 1..14: mol_type = other DNA
  modified_base 6: mod_base = OTHER; note = Locked Nucleic Acid
  modified_base 9: mod_base = OTHER; note = Locked Nucleic Acid
gatcacttggactt

SEQ ID NO: 67 - DNA, 14 bp, synthetic construct
  source 1..14: mol_type = other DNA
  modified_base 3..4: mod_base = OTHER; note = Locked Nucleic Acids
gatcacttggactt

SEQ ID NO: 68 - DNA, 14 bp, synthetic construct
  source 1..14: mol_type = other DNA
gatcacttggactt

SEQ ID NO: 69 - DNA, 14 bp, synthetic construct
  source 1..14: mol_type = other DNA
  modified_base 7..8: mod_base = OTHER; note = Locked Nucleic Acids
gatcacttggactt

SEQ ID NO: 70 - DNA, 14 bp, synthetic construct
  source 1..14: mol_type = other DNA
  modified_base 6..9: mod_base = OTHER; note = Locked Nucleic Acids
gatcacttggactt

SEQ ID NO: 71 - DNA, 14 bp, synthetic construct
  source 1..14: mol_type = other DNA
  modified_base 6: mod_base = OTHER; note = Locked Nucleic Acid
  modified_base 9: mod_base = OTHER; note = Locked Nucleic Acid
gatcacttggactt

SEQ ID NO: 72 - DNA, 14 bp, synthetic construct
  source 1..14: mol_type = other DNA
  modified_base 3..4: mod_base = OTHER; note = Locked Nucleic Acids
gatcacttggactt

SEQ ID NO: 73 - DNA, 14 bp, synthetic construct
  source 1..14: mol_type = other DNA
gatcacttggactt

SEQ ID NO: 74 - DNA, 14 bp, synthetic construct
  source 1..14: mol_type = other DNA
  modified_base 7..8: mod_base = OTHER; note = Locked Nucleic Acids
gatcacttggactt

SEQ ID NO: 75 - DNA, 14 bp, synthetic construct
  source 1..14: mol_type = other DNA
  modified_base 6..9: mod_base = OTHER; note = Locked Nucleic Acids
gatcacttggactt

SEQ ID NO: 76 - DNA, 14 bp, synthetic construct
  source 1..14: mol_type = other DNA
  modified_base 6: mod_base = OTHER; note = Locked Nucleic Acid
  modified_base 9: mod_base = OTHER; note = Locked Nucleic Acid
gatcacttggactt

SEQ ID NO: 77 - DNA, 14 bp, synthetic construct
  source 1..14: mol_type = other DNA
  modified_base 3..4: mod_base = OTHER; note = Locked Nucleic Acids
gatcacttggactt

SEQ ID NO: 78 - DNA, 14 bp, synthetic construct
  source 1..14: mol_type = other DNA
gatcacttggactt

SEQ ID NO: 79 - DNA, 14 bp, synthetic construct
  source 1..14: mol_type = other DNA
  modified_base 7..8: mod_base = OTHER; note = Locked Nucleic Acids
gatcacttggactt

SEQ ID NO: 80 - DNA, 14 bp, synthetic construct
  source 1..14: mol_type = other DNA
  modified_base 6..9: mod_base = OTHER; note = Locked Nucleic Acids
gatcacttggactt

SEQ ID NO: 81 - DNA, 14 bp, synthetic construct
  source 1..14: mol_type = other DNA
  modified_base 6: mod_base = OTHER; note = Locked Nucleic Acid
  modified_base 9: mod_base = OTHER; note = Locked Nucleic Acid
gatcacttggactt

SEQ ID NO: 82 - DNA, 14 bp, synthetic construct
  source 1..14: mol_type = other DNA
  modified_base 3..4: mod_base = OTHER; note = Locked Nucleic Acids
gatcacttggactt

SEQ ID NO: 83 - DNA, 14 bp, synthetic construct
  source 1..14: mol_type = other DNA
gatcacttggactt

SEQ ID NO: 84 - DNA, 14 bp, synthetic construct
  source 1..14: mol_type = other DNA
  modified_base 7..8: mod_base = OTHER; note = Locked Nucleic Acids
gatcacttggactt

SEQ ID NO: 85 - DNA, 14 bp, synthetic construct
  source 1..14: mol_type = other DNA
  modified_base 6..9: mod_base = OTHER; note = Locked Nucleic Acids
gatcacttggactt

SEQ ID NO: 86 - DNA, 14 bp, synthetic construct
  source 1..14: mol_type = other DNA
  modified_base 6: mod_base = OTHER; note = Locked Nucleic Acid
  modified_base 9: mod_base = OTHER; note = Locked Nucleic Acid
gatcacttggactt

SEQ ID NO: 87 - DNA, 14 bp, synthetic construct
  source 1..14: mol_type = other DNA
  modified_base 3..4: mod_base = OTHER; note = Locked Nucleic Acids
gatcacttggactt

SEQ ID NO: 88 - DNA, 14 bp, synthetic construct
  source 1..14: mol_type = other DNA
gatcacttggactt

SEQ ID NO: 89 - DNA, 14 bp, synthetic construct
  source 1..14: mol_type = other DNA
  modified_base 7..8: mod_base = OTHER; note = Locked Nucleic Acids
gatcacttggactt

SEQ ID NO: 90 - DNA, 14 bp, synthetic construct
  source 1..14: mol_type = other DNA
  modified_base 6..9: mod_base = OTHER; note = Locked Nucleic Acids
gatcacttggactt

SEQ ID NO: 91 - DNA, 14 bp, synthetic construct
  source 1..14: mol_type = other DNA
  modified_base 6: mod_base = OTHER; note = Locked Nucleic Acid
  modified_base 9: mod_base = OTHER; note = Locked Nucleic Acid
gatcacttggactt

SEQ ID NO: 92 - DNA, 14 bp, synthetic construct
  source 1..14: mol_type = other DNA
  modified_base 3..4: mod_base = OTHER; note = Locked Nucleic Acids
gatcacttggactt

SEQ ID NO: 93 - DNA, 14 bp, synthetic construct
  source 1..14: mol_type = other DNA
cacagtgcatttca

SEQ ID NO: 94 - DNA, 14 bp, synthetic construct
  source 1..14: mol_type = other DNA
  modified_base 10..11: mod_base = OTHER; note = Locked Nucleic Acids
cacagtgcatttca

SEQ ID NO: 95 - DNA, 14 bp, synthetic construct
  source 1..14: mol_type = other DNA
  modified_base 9..12: mod_base = OTHER; note = Locked Nucleic Acids
cacagtgcatttca

SEQ ID NO: 96 - DNA, 14 bp, synthetic construct
  source 1..14: mol_type = other DNA
  modified_base 9: mod_base = OTHER; note = Locked Nucleic Acid
  modified_base 12: mod_base = OTHER; note = Locked Nucleic Acid
cacagtgcatttca

SEQ ID NO: 97 - DNA, 14 bp, synthetic construct
  source 1..14: mol_type = other DNA
  modified_base 8: mod_base = OTHER; note = Locked Nucleic Acid
  modified_base 13: mod_base = OTHER; note = Locked Nucleic Acid
cacagtgcatttca

SEQ ID NO: 98 - DNA, 14 bp, synthetic construct
  source 1..14: mol_type = other DNA
  modified_base 4: mod_base = OTHER; note = Locked Nucleic Acid
  modified_base 6: mod_base = OTHER; note = Locked Nucleic Acid
cacagtgcatttca

SEQ ID NO: 99 - DNA, 14 bp, synthetic construct
  source 1..14: mol_type = other DNA
cacagtgcatttca

SEQ ID NO: 100 - DNA, 14 bp, synthetic construct
  source 1..14: mol_type = other DNA
  modified_base 10..11: mod_base = OTHER; note = Locked Nucleic Acids
cacagtgcatttca

SEQ ID NO: 101 - DNA, 14 bp, synthetic construct
  source 1..14: mol_type = other DNA
  modified_base 9..12: mod_base = OTHER; note = Locked Nucleic Acids
cacagtgcatttca

SEQ ID NO: 102 - DNA, 14 bp, synthetic construct
  source 1..14: mol_type = other DNA
  modified_base 9: mod_base = OTHER; note = Locked Nucleic Acid
  modified_base 12: mod_base = OTHER; note = Locked Nucleic Acid
cacagtgcatttca

SEQ ID NO: 103 - DNA, 14 bp, synthetic construct
  source 1..14: mol_type = other DNA
  modified_base 8: mod_base = OTHER; note = Locked Nucleic Acid
  modified_base 13: mod_base = OTHER; note = Locked Nucleic Acid
cacagtgcatttca

SEQ ID NO: 104 - DNA, 14 bp, synthetic construct
  source 1..14: mol_type = other DNA
  modified_base 4: mod_base = OTHER; note = Locked Nucleic Acid
  modified_base 6: mod_base = OTHER; note = Locked Nucleic Acid
cacagtgcatttca

SEQ ID NO: 105 - DNA, 12 bp, synthetic construct
  source 1..12: mol_type = other DNA
tcagcgagcacg

SEQ ID NO: 106 - DNA, 12 bp, synthetic construct
  source 1..12: mol_type = other DNA
  modified_base 6..7: mod_base = OTHER; note = Locked Nucleic Acids
tcagcgagcacg

SEQ ID NO: 107 - DNA, 12 bp, synthetic construct
  source 1..12: mol_type = other DNA
  modified_base 5..8: mod_base = OTHER; note = Locked Nucleic Acids
tcagcgagcacg

SEQ ID NO: 108 - DNA, 12 bp, synthetic construct
  source 1..12: mol_type = other DNA
  modified_base 5: mod_base = OTHER; note = Locked Nucleic Acid
  modified_base 8: mod_base = OTHER; note = Locked Nucleic Acid
tcagcgagcacg

SEQ ID NO: 109 - DNA, 12 bp, synthetic construct
  source 1..12: mol_type = other DNA
  modified_base 4: mod_base = OTHER; note = Locked Nucleic Acid
  modified_base 9: mod_base = OTHER; note = Locked Nucleic Acid
tcagcgagcacg

SEQ ID NO: 110 - DNA, 12 bp, synthetic construct
  source 1..12: mol_type = other DNA
tcagcgagcacg

SEQ ID NO: 111 - DNA, 12 bp, synthetic construct
  source 1..12: mol_type = other DNA
  modified_base 5..8: mod_base = OTHER; note = Locked Nucleic Acids
tcagcgagcacg

SEQ ID NO: 112 - DNA, 12 bp, synthetic construct
  source 1..12: mol_type = other DNA
tcagcgagcacg

SEQ ID NO: 113 - DNA, 12 bp, synthetic construct
  source 1..12: mol_type = other DNA
  modified_base 5..8: mod_base = OTHER; note = Locked Nucleic Acids
tcagcgagcacg

SEQ ID NO: 114 - DNA, 14 bp, synthetic construct
  source 1..14: mol_type = other DNA
ttgactactcccat

SEQ ID NO: 115 - DNA, 14 bp, synthetic construct
  source 1..14: mol_type = other DNA
tttgactactccca

SEQ ID NO: 116 - DNA, 14 bp, synthetic construct
  source 1..14: mol_type = other DNA
atttgactactccc

SEQ ID NO: 117 - DNA, 14 bp, synthetic construct
  source 1..14: mol_type = other DNA
catttgactactcc

SEQ ID NO: 118 - DNA, 14 bp, synthetic construct
  source 1..14: mol_type = other DNA
ccatttgactactc

SEQ ID NO: 119 - DNA, 14 bp, synthetic construct
  source 1..14: mol_type = other DNA
cccatttgactact

SEQ ID NO: 120 - DNA, 14 bp, synthetic construct
  source 1..14: mol_type = other DNA
tcccatttgactac

SEQ ID NO: 121 - DNA, 14 bp, synthetic construct
  source 1..14: mol_type = other DNA
ctcccatttgacta

SEQ ID NO: 122 - DNA, 14 bp, synthetic construct
  source 1..14: mol_type = other DNA
actcccatttgact

SEQ ID NO: 123 - DNA, 14 bp, synthetic construct
  source 1..14: mol_type = other DNA
tactcccatttgac

SEQ ID NO: 124 - DNA, 14 bp, synthetic construct
  source 1..14: mol_type = other DNA
ctactcccatttga

SEQ ID NO: 125 - DNA, 14 bp, synthetic construct
  source 1..14: mol_type = other DNA
actactcccatttg

SEQ ID NO: 126 - DNA, 14 bp, synthetic construct
  source 1..14: mol_type = other DNA
gactactcccattt

SEQ ID NO: 127 - DNA, 14 bp, synthetic construct
  source 1..14: mol_type = other DNA
tgactactcccatt

SEQ ID NO: 128 - DNA, 15 bp, synthetic construct
  source 1..15: mol_type = other DNA
tctgactactcccat

SEQ ID NO: 129 - DNA, 15 bp, synthetic construct
  source 1..15: mol_type = other DNA
ttctgactactccca

SEQ ID NO: 130 - DNA, 15 bp, synthetic construct
  source 1..15: mol_type = other DNA
attctgactactccc

SEQ ID NO: 131 - DNA, 15 bp, synthetic construct
  source 1..15: mol_type = other DNA
cattctgactactcc

SEQ ID NO: 132 - DNA, 15 bp, synthetic construct
  source 1..15: mol_type = other DNA
ccattctgactactc

SEQ ID NO: 133 - DNA, 15 bp, synthetic construct
  source 1..15: mol_type = other DNA
cccattctgactact

SEQ ID NO: 134 - DNA, 15 bp, synthetic construct
  source 1..15: mol_type = other DNA
tcccattctgactac

SEQ ID NO: 135 - DNA, 15 bp, synthetic construct
  source 1..15: mol_type = other DNA
ctcccattctgacta

SEQ ID NO: 136 - DNA, 15 bp, synthetic construct
  source 1..15: mol_type = other DNA
actcccattctgact

SEQ ID NO: 137 - DNA, 15 bp, synthetic construct
  source 1..15: mol_type = other DNA
tactcccattctgac

SEQ ID NO: 138 - DNA, 15 bp, synthetic construct
  source 1..15: mol_type = other DNA
ctactcccattctga

SEQ ID NO: 139 - DNA, 15 bp, synthetic construct
  source 1..15: mol_type = other DNA
actactcccattctg

SEQ ID NO: 140 - DNA, 15 bp, synthetic construct
  source 1..15: mol_type = other DNA
gactactcccattct

SEQ ID NO: 141 - DNA, 16 bp, synthetic construct
  source 1..16: mol_type = other DNA
tcctgactactcccat

SEQ ID NO: 142 - DNA, 16 bp, synthetic construct
  source 1..16: mol_type = other DNA
ttcctgactactccca

SEQ ID NO: 143 - DNA, 16 bp, synthetic construct
  source 1..16: mol_type = other DNA
attcctgactactccc

SEQ ID NO: 144 - DNA, 16 bp, synthetic construct
  source 1..16: mol_type = other DNA
cattcctgactactcc

SEQ ID NO: 145 - DNA, 16 bp, synthetic construct
  source 1..16: mol_type = other DNA
ccattcctgactactc

SEQ ID NO: 146 - DNA, 16 bp, synthetic construct
  source 1..16: mol_type = other DNA
cccattcctgactact

SEQ ID NO: 147 - DNA, 16 bp, synthetic construct
  source 1..16: mol_type = other DNA
tcccattcctgactac

SEQ ID NO: 148 - DNA, 16 bp, synthetic construct
  source 1..16: mol_type = other DNA
ctcccattcctgacta

SEQ ID NO: 149 - DNA, 16 bp, synthetic construct
  source 1..16: mol_type = other DNA
actcccattcctgact

SEQ ID NO: 150 - DNA, 16 bp, synthetic construct
  source 1..16: mol_type = other DNA
tactcccattcctgac

SEQ ID NO: 151 - DNA, 16 bp, synthetic construct
  source 1..16: mol_type = other DNA
ctactcccattcctga

SEQ ID NO: 152 - DNA, 16 bp, synthetic construct
  source 1..16: mol_type = other DNA
actactcccattcctg

SEQ ID NO: 153 - DNA, 16 bp, synthetic construct
  source 1..16: mol_type = other DNA
gactactcccattcct

SEQ ID NO: 154 - DNA, 14 bp, synthetic construct
  source 1..14: mol_type = other DNA
  modified_base 14: mod_base = OTHER; note = 3' Minor Groove Binder Moiety
gttcagctcagtgt

SEQ ID NO: 155 - DNA, 14 bp, synthetic construct
  source 1..14: mol_type = other DNA
  modified_base 14: mod_base = OTHER; note = 3' Minor Groove Binder Moiety
tcagtgattattat

SEQ ID NO: 156 - DNA, 14 bp, synthetic construct
  source 1..14: mol_type = other DNA
ataataatcactga

SEQ ID NO: 157 - DNA, 15 bp, synthetic construct
  source 1..15: mol_type = other DNA
actaataatcactga

SEQ ID NO: 158 - DNA, 15 bp, synthetic construct
  source 1..15: mol_type = other DNA
atcaataatcactga

SEQ ID NO: 159 - DNA, 15 bp, synthetic construct
  source 1..15: mol_type = other DNA
atacataatcactga

SEQ ID NO: 160 - DNA, 15 bp, synthetic construct
  source 1..15: mol_type = other DNA
ataactaatcactga

SEQ ID NO: 161 - DNA, 15 bp, synthetic construct
  source 1..15: mol_type = other DNA
ataatcaatcactga

SEQ ID NO: 162 - DNA, 15 bp, synthetic construct
  source 1..15: mol_type = other DNA
ataatacatcactga

SEQ ID NO: 163 - DNA, 15 bp, synthetic construct
  source 1..15: mol_type = other DNA
ataataactcactga

SEQ ID NO: 164 - DNA, 15 bp, synthetic construct
  source 1..15: mol_type = other DNA
ataataatgcactga

SEQ ID NO: 165 - DNA, 15 bp, synthetic construct
  source 1..15: mol_type = other DNA
ataataatctactga

SEQ ID NO: 166 - DNA, 15 bp, synthetic construct
  source 1..15: mol_type = other DNA
ataataatcatctga

SEQ ID NO: 167 - DNA, 15 bp, synthetic construct
  source 1..15: mol_type = other DNA
ataataatcacatga

SEQ ID NO: 168 - DNA, 15 bp, synthetic construct
  source 1..15: mol_type = other DNA
ataataatcactcga

SEQ ID NO: 169 - DNA, 15 bp, synthetic construct
  source 1..15: mol_type = other DNA
ataataatcactgca

SEQ ID NO: 170 - DNA, 17 bp, synthetic construct
  source 1..17: mol_type = other DNA
ataatacccatcactga

SEQ ID NO: 171 - DNA, 17 bp, synthetic construct
  source 1..17: mol_type = other DNA
atacccataatcactga

SEQ ID NO: 172 - DNA, 16 bp, synthetic construct
  source 1..16: mol_type = other DNA
ataataccatcactga

SEQ ID NO: 173 - DNA, 16 bp, synthetic construct
  source 1..16: mol_type = other DNA
ataccataatcactga

SEQ ID NO: 174 - DNA, 15 bp, synthetic construct
  source 1..15: mol_type = other DNA
ataatacatcactga

SEQ ID NO: 175 - DNA, 15 bp, synthetic construct
  source 1..15: mol_type = other DNA
atacataatcactga

SEQ ID NO: 176 - DNA, 15 bp, synthetic construct
  source 1..15: mol_type = other DNA
ataatatatcactga

SEQ ID NO: 177 - DNA, 15 bp, synthetic construct
  source 1..15: mol_type = other DNA
atatataatcactga

SEQ ID NO: 178 - DNA, 15 bp, synthetic construct
  source 1..15: mol_type = other DNA
ataataaatcactga

SEQ ID NO: 179 - DNA, 15 bp, synthetic construct
  source 1..15: mol_type = other DNA
ataaataatcactga

SEQ ID NO: 180 - DNA, 12 bp, synthetic construct
  source 1..12: mol_type = other DNA
gagagcgaccac

SEQ ID NO: 181 - DNA, 12 bp, synthetic construct
  source 1..12: mol_type = other DNA
gagagtgaccac

SEQ ID NO: 182 - DNA, 12 bp, synthetic construct
  source 1..12: mol_type = other DNA
gagagagaccac

SEQ ID NO: 183 - DNA, 12 bp, synthetic construct
  source 1..12: mol_type = other DNA
gagagggaccac

SEQ ID NO: 184 - DNA, 15 bp, synthetic construct
  source 1..15: mol_type = other DNA
aagtccagagtgatc

SEQ ID NO: 185 - DNA, 15 bp, synthetic construct
  source 1..15: mol_type = other DNA
aagtccaaagtgatc

SEQ ID NO: 186 - DNA, 15 bp, synthetic construct
  source 1..15: mol_type = other DNA
aagtccacagtgatc

SEQ ID NO: 187 - DNA, 16 bp, synthetic construct
  source 1..16: mol_type = other DNA
aagtccaccagtgatc

SEQ ID NO: 188 - DNA, 17 bp, synthetic construct
  source 1..17: mol_type = other DNA
aagtccacccagtgatc

SEQ ID NO: 189 - DNA, 18 bp, synthetic construct
  source 1..18: mol_type = other DNA
aagtccaccccagtgatc

SEQ ID NO: 190 - DNA, 16 bp, synthetic construct
  source 1..16: mol_type = other DNA
aagtccaaaagtgatc